(19) United States
(12) Patent Application Publication
(10) Pub. No.: US 2007/0237780 A1
(43) Pub. Date: Oct. 11, 2007

(54) Method of preventing or reducing the risk or incidence of cancer

(76) Inventors: Paul Averback, Beaconsfield (CA); Jack Gemmell, Mississauga (CA)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/684,736

(22) Filed: Mar. 12, 2007

Related U.S. Application Data
(60) Provisional application No. 60/780,829, filed on Mar. 10, 2006.

(51) Int. Cl. A61K 38/10; A61K 39/395

(52) U.S. Cl. 424/178.1; 514/14

(57) ABSTRACT

The Embodiments Disclosed Herein are Directed to Methods of preventing or reducing the risk or incidence of cancer in a tissue, gland, organ, or other cellular mass of a mammal by removing or destroying unwanted cells therefrom using compounds containing or based on peptides described herein.

### CROSS REFERENCE TO RELATED APPLICATION

This application claims benefit of U.S. Provisional Application No. 60/780,829, filed Mar. 10, 2006, which is hereby incorporated by reference in its entirety.

### BACKGROUND

1. Field of the Embodiments

The embodiments include methods of preventing or reducing the risk or incidence of cancer in a tissue, gland, organ, or other cellular mass of a mammal by administering compounds containing or based on peptides described below. The method includes, but is not limited to, administering the compounds intramuscularly, orally, intravenously, intraperitoneally, intracerebrally (intraparenchymally), intracerebroyentricularly, intralesionally, intraocularly, intraarterially, intrathecally, intratumorally, intranasally, topically, transdermally, subcutaneously, or intradermally, either alone or conjugated to a carrier.

2. Description of Related Art

Cancer is the second leading cause of death in the United States. Despite progress to date, the incidence of cancer per 100,000 in the U.S. population has not significantly declined since 1950. Many cancers are difficult to treat, particularly if the cancer has metastasized. One of the most devastating aspects of cancer is the propensity of cells from malignant neoplasms to disseminate from their primary site and metastasize at distant organs. Despite advances in surgical treatment of primary neoplasms and aggressive therapies, most cancer patients die as a result of metastatic disease.

Prevention and risk reduction offer an important strategy in reducing the death toll from cancer. Accordingly, there remains a pressing need for methods of preventing or reducing the risk of acquiring cancer.

Methods of preventing or reducing the risk of acquiring cancer may be directed at the general population at risk or at specific sub-populations that have been identified as being at increased risk as a result of a genetic pre-disposition to certain cancers, life style choices such as tobacco smoking or dietary habits, medical treatments such as immunosuppression, exposure to carcinogenic agents in the environment, such as viruses, chemicals, medications, and radiation, or age-related factors, such as hormone levels.

One method of preventing or reducing the risk of acquiring cancer in a tissue, gland or organ in a subject at risk is to remove or reduce the size of the tissue, gland or organ. For example, women carrying particular genetic mutations of the BRCA1 or BRCA2 gene are at increased risk of developing breast and ovarian cancer. Removal of breasts through prophylactic mastectomy is one strategy used to reduce the risk of breast cancers in those women at high risk as a result of these mutations (Plast Reconstr Surg. 2005; 115:891-909 “Prophylactic mastectomy: indications, options, and reconstructive alternatives”; Cochrane Database Syst Rev. 2004; CD002748 “Prophylactic mastectomy for the prevention of breast cancer”). Many human cancers are thought to involve inherited genetic mutations that increase the lifetime risk of acquiring cancer. Other identified genetic mutations include those to the p53 and RB1 genes. It may be reasonably anticipated that other such genetic mutations will be identified in the future and that removal or reduction in the size of the tissue at risk for cancer for those individuals carrying these specific mutations will be a viable strategy in cancer prevention or risk reduction.

Reduction in the size of tissue, gland or organ may by itself reduce the risk of acquiring cancer. For example, there is evidence that elective breast reduction surgery (reduction mammaplasty) reduces the risk of breast cancer (Plast Reconstr Surg. 2004; 113:2104-10 “Breast reduction surgery and breast cancer risk: does reduction mammaplasty have a role in primary prevention strategies for women at high risk of breast cancer?”; Cancer. 2001; 91:478-83 “Breast cancer risk in relation to amount of tissue removed during breast reduction operations in Sweden.”; Plast Reconstr Surg. 1999; 103:1674-81 “A cohort study of breast cancer risk in breast reduction patients”); and that removal of prostate tissue by TURP (transurethral resection of the prostate) in the treatment of benign prostatic hyperplasia (BPH) reduces the risk of prostate cancer (Cancer 2003; 98:1727-34 “Prostate carcinoma risk subsequent to diagnosis of benign prostatic hyperplasia: a population-based cohort study in Sweden”).

There is an obvious need for an effective agent that will destroy and hence facilitate the prophylactic site-specific removal of or reduction in size of tissues, glands, organs or other cellular masses at risk for cancer, while having mainly local effects and minimal or absent systemic toxicity. Classes of such agents are disclosed in pending U.S. patent application Ser. No. 10/092,934, entitled: Methods of Treating Tumors and Related Conditions Using Neural Thread Proteins, Ser. No. 10/153,334, entitled: Peptides Effective In The Treatment Of Tumors And Other Conditions Requiring The Removal Or Destruction Of Cells; Ser. No. 10/198,069, entitled: Peptides Effective In The Treatment Of Tumors And Other Conditions Requiring The Removal Or Destruction Of Cells; Ser. No. 10/198,070, entitled: Peptides Effective In The Treatment Of Tumors And Other Conditions Requiring The Removal Or Destruction Of Cells, Ser. No. 10/294,891 entitled: Peptides Effective In The Treatment Of Tumors And Other Conditions Requiring The Removal Or Destruction Of Cells; and Ser. No. 10/920,313 entitled: Peptides Effective In The Treatment Of Tumors And Other Conditions Requiring The Removal Or Destruction Of Cells, and pending provisional U.S. patent application Ser. No. 11/680,119 Peptides Effective in the Treatment of Tumors and Other Conditions Requiring the Removal or Destruction of Cells, the disclosures of each of which are incorporated by reference herein in their entirety.

### SUMMARY OF THE EMBODIMENTS

The present embodiments include methods for preventing or reducing the risk of cancer in a subject by administering a compound containing or based on one or more of the peptides described herein (“Specific Peptides”) to a tissue, gland, organ or other cellular mass of a mammal in order to destroy, reduce or remove unwanted cells in the targeted tissue. The method preferably decreases the risk of breast cancer, prostate cancer, ovarian cancer and tonsillar cancer, most preferably prostate cancer. A preferred subject for the methods of the present invention is a human subject at risk of developing cancer, particularly breast cancer, ovarian cancer, prostate cancer, tonsillar cancer, or a combination thereof. In one preferred embodiment, the subject is a man identified as having a relatively high risk of prostate cancer. The high risk of prostate cancer may be based on risk factors such as PSA level.

### DETAILED DESCRIPTION OF THE PREFERRED EMBODIMENTS

Embodiments relate to a method for reducing the risk of cancer in a mammalian subject by removing or reducing the size of tissue, gland, organ or cellular mass at risk for cancer by the administration of a compound containing one or more of the peptides described below (“Specific Peptides”). As used herein, reducing risk or incidence includes decreasing the probability or incidence of an indication, disease, or disorder for a subject compared to a relevant, e.g. untreated, control population, or in the same subject prior to treatment according to the invention. Reduced risk or incidence may include delaying or preventing the onset of an indication, disease, or disorder. Risk or incidence can also be reduced if the severity of an indication, disease, or disorder is reduced to a level such that it is not of clinical relevance. That is, the indication, disease, or disorder may be present but at a level that does not endanger the life, activities, and/or well being of the subject. For example, a small tumor may regress and disappear, or remain static. Preferably tumor formation does not occur. In some circumstances the occurrence of the disorder is reduced to the extent that the subject does not present any signs of the indication, disease, or disorder during and/or after the treatment period.

Terms and phrases used herein are defined as set forth below unless otherwise specified. Throughout this description, the singular forms “a,” “an,” and “the” include plural reference unless the context clearly dictates otherwise.

Specific Peptides

The expression “Specific Peptide” refers to a peptide or other composition of matter claimed in one or more of the following U.S. patent application Ser. No. 10/092,934, entitled: Methods of Treating Tumors and Related Conditions Using Neural Thread Proteins, Ser. No. 10/153,334, entitled: Peptides Effective In The Treatment Of Tumors And Other Conditions Requiring The Removal Or Destruction Of Cells; Ser. No. 10/198,069, entitled: Peptides Effective In The Treatment Of Tumors And Other Conditions Requiring The Removal Or Destruction Of Cells; Ser. No. 10/198,070, entitled: Peptides Effective In The Treatment Of Tumors And Other Conditions Requiring The Removal Or Destruction Of Cells, Ser. No. 10/294,891 entitled: Peptides Effective In The Treatment Of Tumors And Other Conditions Requiring The Removal Or Destruction Of Cells; and Ser. No. 10/920,313 entitled: Peptides Effective In The Treatment Of Tumors And Other Conditions Requiring The Removal Or Destruction Of Cells, and pending provisional U.S. patent application Ser. No. 11/680,119 Peptides Effective in the Treatment of Tumors and Other Conditions Requiring the Removal or Destruction of Cells. The disclosures of each of these applications are incorporated by reference herein in their entirety.

Embodiments of the present invention are premised on the application of Specific Peptides that are capable of removing, destroying and/or killing unwanted cells to the prevention or reduction of cancer risk. Amino acids and amino acid residues described herein may be referred to according to the accepted one or three-letter code provided in the table below.

TABLE 1

| Three-Letter | One-Letter | |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

The expression “Specific Peptide” also includes the specific peptides represented by the following amino acid sequences:

| | |
|---|---|
| 1) | SEQ ID NO.1: |
| | MEFSLLLPRLECNGA or Met-Glu-Phe-Ser-Leu-Leu- |
| | Leu-Pro-Arg-Leu-Glu-Cys-Asn-Gly-Ala |
| 2) | SEQ ID NO.2: |
| | GAISAHRNLRLPGSS or Gly-Ala-Ile-Ser-Ala-His- |
| | Arg-Asn-Leu-Arg-Leu-Pro-Gly-Ser-Ser |
| 3) | SEQ ID NO.3: |
| | DSPASASPVAGITGMCT or Asp-Ser-Pro-Ala-Ser-Ala- |
| | Ser-Pro-Val-Ala-Gly-Ile-Thr-Gly-Met-Cys-Thr |
| 4) | SEQ ID NO.4: |
| | MCTHARLILYFFLVEM or Met-Cys-Thr-His-Ala-Arg- |
| | Leu-Ile-Leu-Tyr-Phe-Phe-Leu-Val-Glu-Met |
| 5) | SEQ ID NO.5: |
| | YFFLVEMEFLH orTyr-Phe-Phe-Leu-Val-Glu-Met- |
| | Glu-Phe-Leu-His |
| 6) | SEQ ID NO.6: |
| | VGQAGLELPTS or Val-Gly-Gln-Ala-Gly-Leu-Glu- |
| | Leu-Pro-Thr-Ser |
| 7) | SEQ ID NO.7: |
| | DDPSVSASQSARYRTGH or Asp-Asp-Pro-Ser-Val-Ser- |
| | Ala-Ser-Gln-Ser-Ala-Arg-Tyr-Arg-Thr-Gly-His |
| 8) | SEQ ID NO.8: |
| | TGHHARLCLANFCG or Thr-Gly-His-His-Ala-Arg- |
| | Leu-Cys-Leu-Ala-Asn-Phe-Cys-Gly |
| 9) | SEQ ID NO.9: |
| | ANFCGRNRVSLMCPSWS or Ala-Asn-Phe-Cys-Gly-Arg- |
| | Asn-Arg-Val-Ser-Leu-Met-Cys-Pro-Ser-Trp-Ser |
| 10) | SEQ ID NO.10: |
| | PELKQSTCLSLPKCWDYRR or Pro-Glu-Leu-Lys-Gln- |
| | Ser-Thr-Cys-Leu-Ser-Leu-Pro-Lys-Cys-Trp-Asp- |
| | Tyr-Arg-Arg |
| 11) | SEQ ID NO.11: |
| | LKQSTCLSLPKCWDYRR or Leu-Lys-Gln-Ser-Thr-Cys- |
| | Leu-Ser-Leu-Pro-Lys-Cys-Trp-Asp-Tyr-Arg-Arg |
| 12) | SEQ ID NO.12: |
| | STCLSLPKCWDYRR or Ser-Thr-Cys-Leu-Ser-Leu- |
| | Pro-Lys-Cys-Trp-Asp-Tyr-Arg-Arg |
| 13) | SEQ ID NO.13: |
| | LSLPKCWDYRR or Leu-Ser-Leu-Pro-Lys-Cys-Trp- |
| | Asp-Tyr-Arg-Arg |
| 14) | SEQ ID NO.14: |
| | KCWDYRRAAVPGL or Lys-Cys-Trp-Asp-Tyr-Arg- |
| | Arg-Ala-Ala-Val-Pro-Gly-Leu |
| 15) | SEQ ID NO.15: |
| | KCWDYRRAAVPGLFILFFL or Lys-Cys-Trp-Asp-Tyr- |
| | Arg-Arg-Ala-Ala-Val-Pro-Gly-Leu-Phe-Ile-Leu- |
| | Phe-Phe-Leu |
| 16) | SEQ ID NO.16: |
| | KCWDYRRAAVPGLFILFFLRHRCP or Lys-Cys-Trp-Asp- |
| | Tyr-Arg-Arg-Ala-Ala-Val-Pro-Gly-Leu-Phe-Ile- |
| | Leu-Phe-Phe-Leu-Arg-His-Arg-Cys-Pro |
| 17) | SEQ ID NO.17: |
| | KCWDYRRAAVPGLFILFFLRHRCPTLTQDEVQWCDHSS or |
| | Lys-Cys-Trp-Asp-Tyr-Arg-Arg-Ala-Ala-Val-Pro- |
| | Gly-Leu-Phe-Ile-Leu-Phe-Phe-Leu-Arg-His-Arg- |
| | Cys-Pro-Thr-Leu-Thr-Gln-Asp-Glu-Val-Gln-Trp- |
| | Cys-Asp-His-Ser-Ser |
| 18) | SEQ ID NO.18: |
| | WDYRR orTrp-Asp-Tyr-Arg-Arg |
| 19) | SEQ ID NO.19: |
| | FILFFLFIHRCPTL or Phe-Ile-Leu-Phe-Phe-Leu- |
| | Arg-His-Arg-Cys-Pro-Thr-Leu |
| 20) | SEQ ID NO.20: |
| | FILFFLRHRCPTLTQDEVQWCDHSS or Phe-Ile-Leu-Phe- |
| | Phe-Leu-Arg-His-Arg-Cys-Pro-Thr-Leu-Thr-Gln- |
| | Asp-Glu-Val-Gln-Trp-Cys-Asp-His-Ser-Ser |
| 21) | SEQ ID NO.21: |
| | HRCPTLTQDEVQWCDHSSLQPSTPEIKHP or His-Arg-Cys- |
| | Pro-Thr-Leu-Thr-Gln-Asp-Glu-Val-Gln-Trp-Cys- |
| | Asp-His-Ser-Ser-Leu-Gln-Pro-Ser-Thr-Pro-Glu- |
| | Ile-Lys-His-Pro |
| 22) | SEQ ID NO.22: |
| | PASASQVAGTKDMH or Pro-Ala-Ser-Ala-Ser-Gln- |
| | Val-Ala-Gly-Thr-Lys-Asp-Met-His |
| 23) | SEQ ID NO.23: |
| | DMHHYTWLIFIFIFNFLR or Asp-Met-His-His-Tyr- |
| | Thr-Trp-Leu-Ile-Phe-Ile-Phe-Ile-Phe-Asn-Phe- |
| | Leu-Arg |
| 24) | SEQ ID NO.24: |
| | HYTWLIFIFIFNFLRQSLN or His-Tyr-Thr-Trp-Leu- |
| | Ile-Phe-Ile-Phe-Ile-Phe-Asn-Phe-Leu-Arg-Gln- |
| | Ser-Leu-Asn |
| 25) | SEQ ID NO.25: |
| | SVTQAGVQWRNLGSLQPLPPGFKLFSCPSLLSSWDYRRPPRLANF |
| | or Ser-Val-Thr-Gln-Ala-Gly-Val-Gln-Trp-Arg- |
| | Asn-Leu-Gly-Ser-Leu-Gln-Pro-Leu-Pro-Pro-Gly- |
| | Phe-Lys-Leu-Phe-Ser-Cys-Pro-Ser-Leu-Leu-Ser- |
| | Ser-Trp-Asp-Tyr-Arg-Arg-Pro-Pro-Arg-Leu-Ala- |
| | Asn-Phe |
| 26) | SEQ ID NO.26: |
| | PGFKLFSCPSLLSSWDYRR or Pro-Gly-Phe-Lys-Leu- |
| | Phe-Ser-Cys-Pro-Ser-Leu-Leu-Ser-Ser-Trp-Asp- |
| | Tyr-Arg-Arg |
| 27) | SEQ ID NO.27: |
| | FKLFSCPSLLSSWDYRRPPRLANF or Phe-Lys-Leu-Phe- |
| | Ser-Cys-Pro-Ser-Leu-Leu-Ser-Ser-Trp-Asp-Tyr- |
| | Arg-Arg-Pro-Pro-Arg-Leu-Ala-Asn-Phe |
| 28) | SEQ ID NO.28: |
| | FSCPSLLSSWDYRR or Phe-Ser-Cys-Pro-Ser-Leu- |
| | Leu-Ser-Ser-Trp-Asp-Tyr-Arg-Arg |
| 29) | SEQ ID NO.29: |
| | SLLSSWDYRR or Ser-Leu-Leu-Ser-Ser-Trp-Asp- |
| | Tyr-Arg-Arg |
| 30) | SEQ ID NO.30: |
| | SSWDY or Ser-Ser-Trp-Asp-Tyr |
| 31) | SEQ ID NO.31: |
| | SSWDYRR or Ser-Ser-Trp-Asp-Tyr-Arg-Arg |
| 32) | SEQ ID NO.32: |
| | SSWDYRRPPRLANFFVFLVEMGFTM or Ser-Ser-Trp-Asp- |
| | Tyr-Arg-Arg-Pro-Pro-Arg-Leu-Ala-Asn-Phe-Phe- |
| | Vat-Phe-Leu-Val-Glu-Met-Gly-Phe-Thr-Met |
| 33) | SEQ ID NO.33: |
| | FVFLVEMGFTM or Phe-Val-Phe-Leu-Val-Glu-Met- |
| | Gly-Phe-Thr-Met |
| 34) | SEQ ID NO.34: |
| | MGFTMFARLILISGPCDLPASAS or Met-Gly-Phe-Thr- |
| | Met-Phe-Ala-Arg-Leu-Ile-Leu-Ile-Ser-Gly-Pro- |
| | Cys-Asp-Leu-Pro-Ala-Ser-Ala-Ser |
| 35) | SEQ ID NO.35: |
| | ISGPC or Ile-Ser-Gly-Pro-Cys |
| 36) | SEQ ID NO.36: |
| | DLPASASQSAGITGVSH or Asp-Leu-Pro-Ala-Ser-Ala- |
| | Ser-Gln-Ser-Ala-Gly-Ile-Thr-Gly-Val-Ser-His |
| 37) | SEQ ID NO.37: |
| | GVSHHARLIFNFCLFEM or Gly-Val-Ser-His-His-Arg- |
| | Leu-Ile-Phe-Asn-Phe-Cys-Leu-Phe-Glu-Met |
| 38) | SEQ ID NO.38: |
| | NFCLFEMESH or Asn-Phe-Cys-Leu-Phe-Glu-Met- |
| | Glu-Ser-His |
| 39) | SEQ ID NO.39: |
| | SVTQAGVQWPNLGSLQPLPPGLKRFSCLSLPSSWDYGHLPPHPAN |
| | F or Ser-Val-Thr-Gln-Ala-Gly-Val-Gln-Trp-Pro- |
| | Asn-Leu-Gly-Ser-Leu-Gln-Pro-Leu-Pro-Pro-Gly- |
| | Leu-Lys-Arg-Phe-Ser-Cys-Leu-Ser-Leu-Pro-Ser- |
| | Ser-Trp-Asp-Tyr-Gly-His-Leu-Pro-Pro-His-Pro- |
| | Ala-Asn-Phe |
| 40) | SEQ ID NO.40: |
| | PPGLKRFSCLSLPSSWDYG or Pro-Pro-Gly-Leu-Lys- |
| | Arg-Phe-Ser-Cys-Leu-Ser-Leu-Pro-Ser-Ser-Trp- |
| | Asp-Tyr-Gly |
| 41) | SEQ ID NO.41: |
| | FSCLSLPSSWDYGH or Phe-Ser-Cys-Leu-Ser-Leu- |
| | Pro-Ser-Ser-Trp-Asp-Tyr-Gly-His |
| 42) | SEQ ID NO.42: |
| | LSLPSSWDY or Leu-Ser-Leu-Pro-Ser-Ser-Trp-Asp- |
| | Tyr-Gly |
| 43) | SEQ ID NO.43: |
| | SSWDYGHLPPHPANFCIFIRGGVSPYLSGWSQTPDLR or Ser- |
| | Ser-Trp-Asp-Tyr-Gly-His-Leu-Pro-Pro-His-Pro- |
| | Ala-Asn-Phe-Cys-Ile-Phe-Ile-Arg-Gly-Gly-Val- |
| | Ser-Pro-Tyr-Leu-Ser-Gly-Trp-Ser-Gln-Thr-Pro- |
| | Asp-Leu-Arg |
| 44) | SEQ ID NO.44: |
| | PGFFKLFSCPSLLSSWDYRR or Pro-Gly-Phe-Phe-Lys- |
| | Leu-Phe-Ser-Cys-Pro-Ser-Leu-Leu-Ser-Ser-Trp- |
| | Asp-Tyr-Arg-Arg |
| 45) | SEQ ID NO.45: |
| | PELKQSTCLSLPKCWDYRR or Pro-Glu-Leu-Lys-Gln- |
| | Ser-Thr-Cys-Leu-Ser-Leu-Pro-Lys-Cys-Trp-Asp- |
| | Tyr-Arg-Arg |
| 46) | SEQ ID NO.46: |
| | PPGLKRFSCLSLPSSWDYG or Pro-Pro-Gly-Leu-Lys- |
| | Arg-Phe-Ser-Cys-Leu-Ser-Leu-Pro-Ser-Ser-Trp- |
| | Asp-Tyr-Gly |
| 47) | SEQ ID NO.47: |
| | FSCLSLPSSWDYGH or Phe-Ser-Cys-Leu-Ser-Leu- |
| | Pro-Ser-Ser-Trp-Asp-Tyr-Gly-His |
| 48) | SEQ ID NO.48: |
| | STCLSLPKCWDYRR or Ser-Thr-Cys-Leu-Ser-Leu- |
| | Pro-Lys-Cys-Trp-Asp-Tyr-Arg-Arg |
| 49) | SEQ ID NO.49: |
| | FSCPSLLSSWDYRR or Phe-Ser-Cys-Pro-Ser-Leu- |
| | Leu-Ser-Ser-Trp-Asp-Tyr-Arg-Arg |
| 50) | SEQ ID NO.50: |
| | LSLPSSWDY or Leu-Ser-Leu-Pro-Ser-Ser-Trp-Asp- |
| | Tyr-Gly |
| 51) | SEQ ID NO.51: |
| | LSLPKCWDYRR or Leu-Ser-Leu-Pro-Lys-Cys-Trp- |
| | Asp-Tyr-Arg-Arg |
| 52) | SEQ ID NO.52: |
| | SLLSSWDYRR or Ser-Leu-Leu-Ser-Ser-Trp-Asp- |
| | Tyr-Arg-Arg |
| 53) | SEQ ID NO.53: |
| | LPSSWDYRR or Leu-Pro-Ser-Ser-Trp-Asp-Tyr-Arg- |
| | Arg |
| 54) | SEQ ID NO.54: |
| | SSWDYRR or Ser-Ser-Trp-Asp-Tyr-Arg-Arg |
| 55) | SEQ ID NO.55: |
| | SSWDY or Ser-Ser-Trp-Asp-Tyr |
| 56) | SEQ ID NO.56: |
| | SSWDYRRFIILFFL or Ser-Ser-Trp-Asp-Tyr-Arg- |
| | Arg-Phe-Ile-Leu-Phe-Phe-Leu |
| 57) | SEQ ID NO.57: |
| | WDYRRFIFNFL or Trp-Asp-Tyr-Arg-Arg-Phe-Ile- |
| | Phe-Asn-Phe-Leu |
| 58) | SEQ ID NO.58: |
| | FNFCLF or Phe-Asn-Phe-Cys-Leu-Phe |
| 59) | SEQ ID NO.59: |
| | FIFNFL or Phe-Ile-Phe-Asn-Phe-Leu |
| 60) | SEQ ID NO.60: |
| | PASASPVAGITGM or Pro-Ala-Ser-Ala-Ser-Pro-Val- |
| | Ala-Gly-Ile-Thr-Gly-Met |
| 61) | SEQ ID NO.61: |
| | PASASQVAGTKDM or Pro-Ala-Ser-Ala-Ser-Gln-Val- |
| | Ala-Gly-Thr-Lys-Asp-Met |
| 62) | SEQ ID NO.62: |
| | PASASQSAGITGV or Pro-Ala-Ser-Ala-Ser-Gln-Ser- |
| | Ala-Gly-Ile-Thr-Gly-Val |
| 63) | SEQ ID NO.63: |
| | PASASPVAG or Pro-Ala-Ser-Ala-Ser-Pro-Val-Ala- |
| | Gly |
| 64) | SEQ ID NO.64: |
| | FFLVEM or Phe-Phe-Leu-Val-Glu-Met |
| 65) | SEQ ID NO.65: |
| | SVTQAGVQW or Ser-Val-Thr-Gln-Ala-Gly-Val-Gln- |
| | Trp |
| 66) | SEQ ID NO.66: |
| | IDQQVLSRIKLEIKRCL or Ile-Asp-Gln-Gln-Val-Leu- |
| | Ser-Arg-Ile-Lys-Leu-Glu-Ile-Lys-Arg-Cys-Leu |
| 67) | SEQ ID NO.67: |
| | LSRIKLEIK or Leu-Ser-Arg-Ile-Lys-Leu-Glu- |
| | Ile-Lys |
| 68) | SEQ ID NO.68: |
| | GDHGRPNLSRLKLAIKYEVKKM or Gly-Asp-His-Gly- |
| | Arg-Pro-Asn-Leu-Ser-Arg-Leu-Lys-Leu-Ala-Ile- |
| | Lys-Tyr-Glu-Val-Lys-Lys-Met |
| 69) | SEQ ID NO.69: |
| | QQSIAVKFLAVFGVSI or Gln-Gln-Ser-Ile-Ala-Val- |
| | Lys-Phe-Leu-Ala-Val-Phe-Gly-Val-Ser-Ile |
| 70) | SEQ ID NO.70: |
| | GLLFPVFSVCYLIAPKSPLGL or Gly-Leu-Leu-Phe-Pro- |
| | Val-Phe-Ser-Val-Cys-Tyr-Leu-Ile-Ala-Pro-Lys- |
| | Ser-Pro-Leu-Gly-Leu |
| 71) | SEQ ID NO.71: |
| | MMVCWNREGKWVYFI or Met-Met-Val-Cys-Trp-Asn- |
| | Arg-Phe-Gly-Lys-Trp-Val-Tyr-Phe-Ile |
| 72) | SEQ ID NO.72: |
| | SAIFNFGPRYLYHGV or Ser-Ala-Ile-Phe-Asn-Phe- |
| | Gly-Pro-Arg-Tyr-Leu-Tyr-His-Gly-Val |
| 73) | SEQ ID NO.73: |
| | PFYFLILVRIISFLI or Pro-Phe-Tyr-Phe-Leu-Ile- |
| | Leu-Val-Arg-Ile-Ile-Ser-Phe-Leu-Ile |
| 74) | SEQ ID NO.74: |
| | GDMEDVLLNCTLLKR or Gly-Asp-Met-Glu-Asp-Val- |
| | Leu-Leu-Asn-Cys-Thr-Leu-Leu-Lys-Arg |
| 75) | SEQ ID NO.75: |
| | SSRFREWGALVCSMD or Ser-Ser-Arg-Phe-Arg-Phe- |
| | Trp-Gly-Ala-Leu-Val-Cys-Ser-Met-Asp |
| 76) | SEQ ID NO.76: |
| | SCRFSRVAVTYRFIT or Ser-Cys-Arg-Phe-Ser-Arg- |
| | Val-Ala-Val-Thr-Tyr-Arg-Phe-Ile-Thr |
| 77) | SEQ ID NO.77: |
| | LLNIPSPAVWMARNT or Leu-Leu-Asn-Ile-Pro-Ser- |
| | Pro-Ala-Val-Trp-Met-Ala-Arg-Asn-Thr |
| 78) | SEQ ID NO.78: |
| | MAQSRLTATSASRVQ or Met-Ala-Gln-Ser-Arg-Leu- |
| | Thr-Ala-The-Ser-Ala-Ser-Arg-Val-Gln |
| 79) | SEQ ID NO.79: |
| | AILLSQPPKQLGLRA or Ala-Ile-Leu-Leu-Ser-Gln- |
| | Pro-Pro-Lys-Gln-Leu-Gly-Leu-Arg-Ala |
| 80) | SEQ ID NO.80: |
| | PANTPLIFVFSLEAG or Pro-Ala-Asn-Thr-Pro-Leu- |
| | Ile-Phe-Val-Phe-Ser-Leu-Glu-Ala-Gly |
| 81) | SEQ ID NO.81: |
| | FHHICQAGLKLLTSG or Phe-His-His-Ile-Cys-Gln- |
| | Ala-Gly-Leu-Lys-Leu-Leu-Thr-Ser-Gly |
| 82) | SEQ ID NO.82: |
| | DPPASAFQSAGITGV or Asp-Pro-Pro-Ala-Ser-Ala- |
| | Phe-Gln-Ser-Ala-Gly-Ile-Thr-Gly-Val |
| 83) | SEQ ID NO.83: |
| | SHLTQPANLDKKICS or Ser-His-Leu-Thr-Gln-Pro- |
| | Ala-Asn-Leu-Asp-Lys-Lys-Ile-Gys-Ser |
| 84) | SEQ ID NO.84: |
| | NGGSCYVAQAGLKLLASCNPSK or Asn-Gly-Gly-Ser- |
| | Cys-Tyr-Val-Ala-Gln-Ala-Cly-Leu-Lys-Leu-Leu- |
| | Ala-Ser-Cys-Asn-Pro-Ser-Lys |
| 85) | SEQ ID NO.85: |
| | MWTLKSSLVLLLCLT or Met-Trp-Thr-Leu-Lys-Ser- |
| | Ser-Leu-Val-Leu-Leu-Leu-Cys-Leu-Thr |
| 86) | SEQ ID NO.86: |
| | CSYAFMFSSLRQKTS or Cys-Ser-Tyr-Ala-Phe-Met- |
| | Phe-Ser-Ser-Leu-Arg-Gln-Lys-Thr-Ser |
| 87) | SEQ ID NO.87: |
| | EPQGKVPCGEHFRTR or Glu-Pro-Gln-Gly-Lys-Val- |
| | Pro-Cys-Gly-Glu-His-Phe-Arg-Ile-Arg |
| 88) | SEQ ID NO.88: |
| | QNLPEHTQGWLGSKW or Gln-Asn-Leu-Pro-Glu-His- |
| | Thr-Gln-Gly-Trp-Leu-Gly-Ser-Lys-Trp |
| 89) | SEQ ID NO.89: |
| | LWLLFAVVPFVILKC or Leu-Trp-Leu-Leu-Phe-Ala- |
| | Val-Val-Pro-Phe-Val-Ile-Leu-Lys-Cys |
| 90) | SEQ ID NO.90: |
| | QRDSEKNKVRMAPFF or Gln-Arg-Asp-Ser-Glu-Lys- |
| | Asn-Lys-Val-Arg-Met-Ala-Pro-Phe-Phe |
| 91) | SEQ ID NO.91: |
| | LHHIDSISGVSGKRMF or Leu-His-His-Ile-Asp-Ser- |
| | Ile-Ser-Gly-Val-Ser-Gly-Lys-Arg-Met-Phe |
| 92) | SEQ ID NO.92: |
| | EAYYTMLHLPTTNRP or Glu-Ala-Tyr-Tyr-Thr-Met- |
| | Leu-His-Leu-Pro-Thr-Thr-Asn-Arg-Pro |
| 93) | SEQ ID NO.93: |
| | KIAHCILFNQPHSPR or Lys-Ile-Ala-His-Cys-Ile- |
| | Leu-Phe-Asn-Gln-Pro-His-Ser-Pro-Arg |
| 94) | SEQ ID NO.94: |
| | SNSHSHPNPLKLHRR or Ser-Asn-Ser-His-Ser-His- |
| | Pro-Asn-Pro-Leu-Lys-Leu-His-Arg-Arg |
| 95) | SEQ ID NO.95: |
| | SHSHNRPRAYILITI or Ser-His-Ser-His-Asn-Arg- |
| | Pro-Arg-Ala-Tyr-Ile-Leu-Ile-Thr-Ile |
| 96) | SEQ ID NO.96: |
| | LPSKLKLRTHSQSHH or Leu-Pro-Ser-Lys-Leu-Lys- |
| | Leu-Arg-Thr-His-Ser-Gln-Ser-His-His |
| 97) | SEQ ID NO.97: |
| | NPLSRTSNSTPTNSFLMTSSKPR or Asn-Pro-Leu-Ser- |
| | Arg-Thr-Ser-Asn-Ser-Thr-Pro-Thr-Asn-Ser-Phe- |
| | Leu-Met-Thr-Ser-Ser-Lys-Pro-Arg |
| 98) | SEQ ID NO.98: |
| | SSSLGLPKOWDYRHF or Ser-Ser-Ser-Leu-Gly-Leu- |
| | Pro-Lys-Cys-Trp-Asp-Tyr-Arg-His-Glu |
| 99) | SEQ ID NO.99: |
| | LLSLALMINFRVMAC or Leu-Leu-Ser-Leu-Ala-Leu- |
| | Met-Ile-Asn-Phe-Arg-Val-Met-Ala-Cys |
| 100) | SEQ ID NO.100: |
| | TFKQHIELRQKISIV or Thr-Phe-Lys-Gln-His-Ile- |
| | Glu-Leu-Arg-Gln-Lys-Ile-Ser-Ile-Val |
| 101) | SEQ ID NO.101: |
| | PRKLCCMGPVCPVKI or Pro-Arg-Lys-Leu-Cys-Cys- |
| | Met-Gly-Pro-Val-Cys-Pro-Val-Lys-Ile |
| 102) | SEQ ID NO.102: |
| | ALLTINGHCTWLPAS or Ala-Leu-Leu-Thr-Ile-Asn- |
| | Gly-His-Cys-Thr-Trp-Leu-Pro-Ala-Ser |
| 103) | SEQ ID NO.103: |
| | MFVFCLILNREKIKG or Met-Phe-Val-Phe-Cys-Leu- |
| | Ile-Leu-Asn-Arg-Glu-Lys-Ile-Lys-Gly |
| 104) | SEQ ID NO.104: |
| | GNSSFFLLSFFFSFQ or Gly-Asn-Ser-Ser-Phe-Phe- |
| | Leu-Leu-Ser-Phe-Phe-Phe-Ser-Phe-Gln |
| 105) | SEQ ID NO.105: |
| | NCCQCFQCRTTEGYA or Asn-Cys-Cys-Gln-Cys-Phe- |
| | Gln-Cys-Arg-Thr-Thr-Glu-Gly-Tyr-Ala |
| 106) | SEQ ID NO.106: |
| | VFCFYCLVDKAAFECWWFYSFDT or Val-Glu-Cys-Phe- |
| | Tyr-Cys-Leu-Val-Asp-Lys-Ala-Ala-Phe-Glu-Cys- |
| | Trp-Trp-Phe-Tyr-Ser-Phe-Asp-Thr |
| 107) | SEQ ID NO.107: |
| | MEPHTVAQAGVPQHD or Met-Glu-Pro-His-Thr-Val- |
| | Ala-Gln-Ala-Gly-Val-Pro-Gln-His-Asp |
| 108) | SEQ ID NO.108: |
| | LGSLQSLLPRFKRFS or Leu-Gly-Ser-Leu-Gln-Ser- |
| | Leu-Leu-Pro-Arg-Phe-Lys-Arg-Phe-Ser |
| 109) | SEQ ID NO.109: |
| | CLILPKIWDYRNMNT or Cys-Leu-Ile-Leu-Pro-Lys- |
| | Ile-Trp-Asp-Tyr-Arg-Asn-Met-Asn-Thr |
| 110) | SEQ ID NO.110: |
| | ALIKRNRYTPETGRKS or Ala-Leu-Ile-Lys-Arg-Asn- |
| | Arg-Tyr-Thr-Pro-Glu-Thr-Gly-Arg-Lys-Ser |
| 111) | SEQ ID NO.111: |
| | IDQQVLSRI or Ile-Asp-Gln-Gln-Val-Leu-Ser-Arg- |
| | Ile |
| 112) | SEQ ID NO.112: |
| | KLEIKRCL or Lys-Leu-Glu-Ile-Lys-Arg-Cys-Leu |
| 113) | SEQ ID NO.113: |
| | VLSRIK or Val-Leu-Ser-Arg-Ile-Lys |
| 114) | SEQ ID NO.114: |
| | RIKLEIK or Arg-Ile-Lys-Leu-Glu-Ile-Lys |
| 115) | SEQ ID NO.115: |
| | VLSRIKLEIKRCL or Val-Leu-Ser-Arg-Ile-Lys-Leu- |
| | Glu-Ile-Lys-Arg-Cys-Leu; |
| and | |
| 116) | SEQ ID NO.116: |
| | IDQQVLSRIKLEI or Ile-Asp-Gln-Gln-Val-Leu-Ser- |
| | Arg-Ile-Lys-Leu-Glu-Ile. |

The expression “Specific Peptide” as it is used herein refers to the peptides listed above, and includes homologues, derivatives, variants, fusion proteins, and peptide mimetics of such peptides unless the context indicates otherwise. The expression “Specific Peptide” also includes (but is not limited to) the peptides specifically listed in U.S. patent application Ser. No. 10/092,934, Ser. No. 10/153,334, Ser. No. 10/198,069, Ser. No. 10/198,070, Ser. No. 10/294,891, Ser. No. 10/920,313, and Ser. No. 11/680,119.

The term “fragment” refers to a protein or polypeptide that consists of a continuous subsequence of the amino acid sequence of a protein or peptide and includes naturally occurring fragments such as splice variants and fragments resulting from naturally occurring in vivo protease activity. Such a fragment may be truncated at the amino terminus, the carboxy terminus, and/or internally (such as by natural splicing). Such fragments may be prepared with or without an amino terminal methionine. The term “fragment” includes fragments, whether identical or different, from the same protein or peptide, with a contiguous amino acid sequence in common or not, joined together, either directly or through a linker. The skilled artisan also will be capable of selecting a suitable fragment for use in the embodiments without undue experimentation using the guidelines and procedures outlined herein.

The term “variant” refers to a protein or polypeptide in which one or more amino acid substitutions, deletions, and/or insertions are present as compared to the amino acid sequence of an protein or peptide and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in Table 2 below.

TABLE 2
Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagine |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

Table 3 sets out another scheme of amino acid substitution:

TABLE 3

| Original Residue | Substitutions |
|---|---|
| Ala | gly; ser |
| Arg | lys |
| Asn | gln; his |
| Asp | glu |
| Cys | ser |
| Gln | asn |
| Glu | asp |
| Gly | ala; pro |
| His | asn; gln |
| Ile | eu; val |
| Leu | ile; val |
| Lys | arg; gln; glu |
| Met | leu; tyr; ile |
| Phe | met; leu; tyr |
| Ser | thr |
| Thr | ser |
| Trp | tyr |
| Tyr | trp; phe |
| Val | ile; leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. For example, a cysteine residue may be added at both the amino and carboxy terminals of a Specific Peptide in order to allow the cyclisation of the Peptide by the formation of a di-sulphide bond. The term “variant” also encompasses polypeptides that have the amino acid sequence of the Specific Peptide with at least one and up to 25 or more additional amino acids flanking either the 3′ or 5′ end of the peptide.

The term “derivative” refers to a chemically modified protein or polypeptide that has been chemically modified either by natural processes, such as processing and other post-translational modifications, but also by chemical modification techniques, as for example, by addition of one or more polyethylene glycol molecules, sugars, phosphates, and/or other such molecules, where the molecule or molecules are not naturally attached to wild-type proteins or Specific Peptides. Derivatives include salts. Such chemical modifications are well described in basic texts and in more detailed monographs, as well as in a voluminous research literature, and they are well known to those of skill in the art. It will be appreciated that the same type of modification may be present in the same or varying degree at several sites in a given protein or polypeptide. Also, a given protein or polypeptide may contain many types of modifications. Modifications can occur anywhere in a protein or polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2nd Ed., T. E. Creighton, W. H. Freeman and Company, New York (1993) and Wold, F., “Posttranslational Protein Modifications: Perspectives and Prospects,” pgs. 1-12 in Posttransiational Covalent Modification Of Proteins, B. C. Johnson, Ed., Academic Press, New York (1983); Seifter et al., Meth. Enzymol. 182:626-646 (1990) and Rattan et al., “Protein Synthesis: Posttranslational Modifications and Aging,” Ann. N.Y. Acad. Sci. 663: 48-62 (1992). The term “derivatives” include chemical modifications resulting in the protein or polypeptide becoming branched or cyclic, with or without branching. Cyclic, branched and branched circular proteins or polypeptides may result from post-translational natural processes and may be made by entirely synthetic methods, as well.

The term “homologue” refers to a protein that is at least 60 percent identical in its amino acid sequence of a Specific Peptide as determined by standard methods that are commonly used to compare the similarity in position of the amino acids of two polypeptides. The degree of similarity or identity between two proteins can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M Stockton Press, New York, 1991; and Carillo H. and Lipman, D., SIAM, J. Applied Math., 48:1073 (1988). Preferred methods to determine identity are designed to give the largest match between the sequences tested. Methods to determine identity and similarity are codified in publicly available computer programs.

Preferred computer program methods useful in determining the identity and similarity between two sequences include, but are not limited to, the GCG program package (Devereux, J., et al., Nucleic Acids Research, 12(1): 387 (1984)), BLASTP, BLASTN, and FASTA, Atschul, S. F. et al., J. Molec. Biol., 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol., 215: 403-410 (1990). By way of example, using a computer algorithm such as GAP (Genetic Computer Group, University of Wisconsin, Madison, Wis.), the two proteins or polypeptides for which the percent sequence identity is to be determined are aligned for optimal matching of their respective amino acids (the “matched span”, as determined by the algorithm).

A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al. in: Atlas of Protein Sequence and Structure, vol. 5, supp. 3 for the PAM250 comparison matrix; see Henikoff et al., Proc. Natl. Acad. Sci. USA, 89:10915-10919 for the BLOSUM 62 comparison matrix) also may be used by the algorithm. The percent identity then is calculated by the algorithm. Homologues will typically have one or more amino acid substitutions, deletions, and/or insertions as compared with the comparison protein or peptide, as the case may be.

The term “fusion protein” refers to a protein where one or more peptides are recombinantly fused or chemically conjugated (including covalently and non-covalently) to a protein such as (but not limited to) an antibody or antibody fragment like an F.sub.ab fragment or short chain Fv. The term “fusion protein” also refers to multimers (i.e. dimers, trimers, tetramers and higher multimers) of peptides. Such multimers comprise homomeric multimers comprising one peptide, heteromeric multimers comprising more than one peptide, and heteromeric multimers comprising at least one peptide and at least one other protein. Such multimers may be the result of hydrophobic, hyrdrophilic, ionic and/or covalent associations, bonds or links, may be formed by cross-links using linker molecules or may be linked indirectly by, for example, liposome formation

The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. Examples of peptide mimetics in this broader sense (where part of a peptide is replaced by a structure lacking peptide bonds) are described below. Whether completely or partially non-peptide, peptide mimetics according to the embodiments provide a spatial arrangement of reactive chemical moieties that closely resemble the three-dimensional arrangement of active groups in the peptide on which the peptide mimetic is based. As a result of this similar active-site geometry, the peptide mimetic has effects on biological systems that are similar to the biological activity of the peptide.

The peptide mimetics of the embodiments are preferably substantially similar in both three-dimensional shape and biological activity to the peptides described herein. Examples of methods of structurally modifying a peptide known in the art to create a peptide mimetic include the inversion of backbone chiral centers leading to D-amino acid residue structures that may, particularly at the N-terminus, lead to enhanced stability for proteolytical degradation without adversely affecting activity. An example is given in the paper “Tritriated D-ala.sup.1-Peptide T Binding”, Smith C. S. et al., Drug Development Res., 15, pp. 371-379 (1988). A second method is altering cyclic structure for stability, such as N to C interchain imides and lactames (Ede et al. in Smith and Rivier (Eds.) “Peptides: Chemistry and Biology”, Escom, Leiden (1991), pp. 268-270). An example of this is given in conformationally restricted thymopentin-like compounds, such as those disclosed in U.S. Pat. No. 4,457,489 (1985), Goldstein, G. et al., the disclosure of which is incorporated by reference herein in its entirety. A third method is to substitute peptide bonds in the peptide by pseudopeptide bonds that confer resistance to proteolysis.

A number of pseudopeptide bonds have been described that in general do not affect peptide structure and biological activity. One example of this approach is to substitute retro-inverso pseudopeptide bonds (“Biologically active retroinverso analogues of thymopentin”, Sisto A. et al in Rivier, J. E. and Marshall, G. R. (eds) “Peptides, Chemistry, Structure and Biology”, Escom, Leiden (1990), pp. 722-773) and Dalpozzo, et al. (1993), Int. J. Peptide Protein Res., 41:561-566, incorporated herein by reference). According to this modification, the amino acid sequences of the peptides may be identical to the sequences of an peptide described above, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus. Further modifications also can be made by replacing chemical groups of the amino acids with other chemical groups of similar structure. Another suitable pseudopeptide bond that is known to enhance stability to enzymatic cleavage with no or little loss of biological activity is the reduced isostere pseudopeptide bond (Couder, et al. (1993), Int. J. Peptide Protein Res., 41:181-184, incorporated herein by reference in its entirety).

Thus, the amino acid sequences of these peptides may be identical to the sequences of a Specific Peptide, except that one or more of the peptide bonds are replaced by an isostere pseudopeptide bond. Preferably the most N-terminal peptide bond is substituted, since such a substitution would confer resistance to proteolysis by exopeptidases acting on the N-terminus. The synthesis of peptides with one or more reduced isostere pseudopeptide bonds is known in the art (Couder, et al. (1993), cited above). Other examples include the introduction of ketomethylene or methylsulfide bonds to replace peptide bonds.

Peptoid derivatives of peptides represent another class of peptide mimetics that retain the important structural determinants for biological activity, yet eliminate the peptide bonds, thereby conferring resistance to proteolysis (Simon, et al., 1992, Proc. Natl. Acad. Sci. USA, 89:9367-9371, incorporated herein by reference in its entirety). Peptoids are oligomers of N-substituted glycines. A number of N-alkyl groups have been described, each corresponding to the side chain of a natural amino acid (Simon, et al. (1992), cited above). Some or all of the amino acids of the peptides may be replaced with the N-substituted glycine corresponding to the replaced amino acid.

The expression “peptide mimetic” or “mimetic” also includes reverse-D peptides and enantiomers as defined below.

The term “reverse-D peptide” refers to a biologically active protein or peptide consisting of D-amino acids arranged in a reverse order as compared to the L-amino acid sequence of an peptide. Thus, the carboxy terminal residue of an L-amino acid peptide becomes the amino terminal for the D-amino acid peptide and so forth. For example, the peptide, ETESH, becomes H_{d}S_{d}E_{d}T_{d}E_{d}, where E_{d}, H_{d}, S_{d}, and T_{d} are the D-amino acids corresponding to the L-amino acids, E, H, S, and T respectively.

The term “enantiomer” refers to a biologically active protein or peptide where one or more the L-amino acid residues in the amino acid sequence of an peptide is replaced with the corresponding D-amino acid residue(s).

A “composition” as used herein, refers broadly to any composition containing a recited peptide or amino acid sequence. The composition may comprise a dry formulation, an aqueous solution, or a sterile composition. Compositions comprising peptides may be employed as hybridization probes. The probes may be stored in freeze-dried form and may be associated with a stabilizing agent such as a carbohydrate. In hybridizations, the probe may be deployed in an aqueous solution containing salts, e.g., NaCl, detergents, e.g., sodium dodecyl sulfate (SDS), and other components, e.g., Denhardt's solution, dry milk, salmon sperm DNA, etc.

Cancer

Cancer is an abnormality in a cell's internal regulatory mechanisms that results in uncontrolled growth and reproduction of the cell. Normal cells make up tissues, and when these cells lose their ability to behave as a specified, controlled, and coordinated unit in a process known as dedifferentiation, the defect leads to disarray amongst the cell population. When this occurs, a tumor is formed. If left untreated, a cancer typically invades other tissues, spreads, and eventually results in death. By reducing the incidence of cancer, the embodiments of the present invention prevent or reduce the likelihood of this invasion, spread, and death.

As used herein, the term “cancer” includes any cellular tumor, or mass, that, when not treated, grows, and includes, for example, tumors of lung, breast, stomach, pancreas, prostate, bladder, bone, ovary, skin, kidney, sinus, colon, intestine, stomach, rectum, esophagus, blood, brain and its coverings, spinal cord and its coverings, muscle, connective tissue, adrenal, parathyroid, thyroid, uterus, testis, pituitary, reproductive organs, liver, gall bladder, eye, ear, nose, throat, tonsils, mouth, lymph nodes and lymphoid system, and other organs. The term “cancer” also is intended to encompass all forms of human carcinomas, sarcomas and melanomas which occur in the poorly differentiated, moderately differentiated, and well-differentiated forms.

Subjects Identified as Being at Risk for Cancer

Cancers can arise from a variety of causes, and the present embodiments may be effective in reducing risk of cancers in subjects having such risk factors as, for example, genetic predisposition, hormonal levels, increased age, family history of cancer, lifestyle choices such as cigarette smoking, exposure to chemical carcinogens, immunosuppressive treatment, viral infection, or radiation exposure.

Many cancers are thought to involve genetic mutations that result in, for example, the conversion of protooncogenes to oncogenes and/or dysfunction of tumor suppressor genes. The present embodiments are directed, for example, to reducing the risk of breast cancer in subjects carrying mutations associated with breast cancer, such as mutations of the BRCA1 or BRCA2 gene.

Prostate Cancer

Prostate cancer is the most commonly diagnosed cancer and the second leading cause of cancer death for men in the United States. The American Cancer Society estimates that in 2006, 234,460 men will be diagnosed with prostate cancer and 27,350 will die of this disease. The present embodiments include methods for administering a Specific Peptide to a man identified through risk factors, physical examination, genetic testing and/or abnormal biomarker values as being at an elevated risk for prostate cancer.

A number of risk factors have been linked to increased risk of a man developing prostate cancer. According to the American Cancer Society, these include:

(1) Age: The chance of getting prostate cancer goes up as a man gets older. About 2 out of every 3 prostate cancers are found in men over the age of 65.

(2) Race: For unknown reasons, prostate cancer is more common among African-American men than among white men. And African-American men are twice as likely to die of the disease. Prostate cancer occurs less often in Asian men than in whites.

(3) Nationality: Prostate cancer is most common in North America and northwestern Europe. It is less common in Asia, Africa, Central and South America.

(4) Family history: Men with close family members (father or brother) who have had prostate cancer are more likely to get it themselves, especially if their relatives were young when they got the disease.

Evidence has been found suggesting a link between prostate cancer and higher levels of androgens and elevated serum levels of insulin-like growth factor 1 (IGF-1). But others have not found such a link. More research is needed in this area.

Risk of prostate cancer can also be determined by abnormal levels of biomarkers such as prostate specific antigen (PSA) and p53, p21, p27, and E-cadherin. These markers may be used with other factors such as prostate volume as determined by digital rectal examination (DRE), transrectal ultrasound (TRUS) or other means, age, and race to predict elevated risk of prostate cancer.

Breast Cancer

Breast cancer is the most common cancer among women, other than skin cancer, and the second leading cause of cancer death in women, after lung cancer. According to the American Cancer Society, an estimated 212,920 women in the United States will be found to have invasive breast cancer in 2006 and about 40,970 women will die from it.

The methods of preferred embodiments encompass the administration of a Specific Peptide to an individual, particularly a woman, identified through risk factors, physical examination, genetic testing and/or abnormal biomarker values as being at an elevated risk for breast cancer.

A number of risk factors have been linked to increased risk of a person developing breast cancer. According to the American Cancer Society, these include:

(1) Gender: Being a woman is the main risk for breast cancer. While men can also get the disease, it is about 100 times more common in women than in men.

(2) Age: The chance of getting breast cancer goes up as a woman gets older. Nearly 8 out of 10 breast cancers are found in women over age 50.

(3) Genetic risk factors: About 5% to 10% of breast cancers are linked to changes (mutations) in certain genes. The most common gene changes are those of the BRCA1 and BRCA2 genes. Women with these gene changes have up to an 80% chance of getting breast cancer during their lifetimes.

(4) Family history: Breast cancer risk is higher among women whose close blood relatives have this disease.

(5) Personal history of breast cancer: A woman with cancer in one breast has a greater chance of getting a new cancer in the other breast or in another part of the same breast.

(6) Race: White women are slightly more likely to get breast cancer than are African-American women.

(7) Earlier abnormal breast biopsy: Certain types of abnormal biopsy results can be linked to a slightly higher risk of breast cancer.

(8) Earlier breast radiation: Women who have had radiation treatment to the chest area earlier in life have a greatly increased risk of breast cancer.

(9) Menstrual periods: Women who began having periods early (before 12 years of age) or who went through menopause after the age of 55 have a slightly increased risk of breast cancer.

(10) Treatment with DES: In the past, some pregnant women were given the drug DES (diethylstilbestrol) because it was thought to lower their chances of losing the baby. Recent studies have shown that these women have a slightly increased risk of getting breast cancer.

(11) Not having children: Women who have had not had children, or who had their first child after age 30, have a slightly higher risk of breast cancer.

(12) Birth control pills: Studies have found that women now using birth control pills have a slightly greater risk of breast cancer.

(13) Hormone replacement therapy (HRT): Long-term use (several years or more) of combined HRT (estrogens together with progesterone) after menopause increases the risk of breast cancer.

(14) Alcohol: Use of alcohol is clearly linked to a slightly increased risk of getting breast cancer. Women who have 1 drink a day have a very small increased risk. Those who have 2 to 5 drinks daily have about 1½ times the risk of women who drink no alcohol.

(15) Diet: Being overweight is linked to a higher risk of breast cancer, especially for post-menopausal women and if the weight gain took place during adulthood.

(16) Smoking: While a direct link between smoking and breast cancer has not been found, some studies suggest it might increase breast cancer risk, particularly for women who start smoking as teens.

The methods provided herein may benefit a subject at risk of developing breast cancer by reducing the probability that they get cancer. The methods provided herein may reduce the risk of various types of breast cancer, for example, those cancers due to or correlated with a genetic mutation in a tumor suppressor gene, e.g. p 53, BRCA1, BRCA2 and the like. Risk of breast cancer of sporadic origin, or due to, for example, increased age, family history of cancer, exposure to chemical carcinogens, an immunosuppressive drug, viral infection, or physical factors such as radiation can also be reduced by the method in accordance with the embodiments.

More particularly, populations considered to be in need of treatment according to the present embodiments to reduce high risk of breast cancer can be defined using the Gail model, Gail M H, Brinton L A, Byar D P et al. (1989), and other models which estimate cancer risk based on risk factors.

Methods in accordance to preferred embodiments encompass the administration of Specific Peptides to persons with elevated sex hormone levels indicative of increased risk of breast cancer. Estrogen is thought to play a role in the etiology of certain breast cancers. For instance, see Cauley J A Lucas F L, Kuller L H, Stone K, Browner W, Cummings S R (1999). In particular, elevated serum estradiol and testosterone concentrations are associated with a high risk of breast cancer. Study of Osteoporotic Fractures Research Group. Annals of Inter Med. 130:270-277. The authors found that the relative risk for breast cancer in women with the highest concentration of bioavailable estradiol (≧6.83 pmol/L or 1.9 pg/ml) was 3.6 (95% Cl, 1.3 to 10.0) compared with women with the lowest concentration. The risk for breast cancer in women with the highest concentration of free testosterone compared with those with the lowest concentration was 3.3 (Cl, 1.1 to 10.3). The estimated incidence of breast cancer per 1000 person-years was 0.4 (Cl, 0.0 to 1.3) in women with the lowest levels of bioavailable estradiol and free testosterone compared with 6.5 (Cl, 2.7 to 10.3) in women with the highest concentrations of these hormones.

Risk factors for other cancers, such as oropharyneal carcinoma, thyroid cancer, lymphoma, lung cancer, colon cancer and stomach cancer, are well known to those skilled in the arts and described in the relevant scientific and medical literature.

Method of Administration

The method of one embodiment comprises administering a Specific Peptide by direct or indirect infusion of Peptide into the tissue, gland, organ or cellular mass to be treated. One example of such an embodiment is the direct injection of Peptide into the tissue to be treated. The treatment may consist of a single injection, multiple injections on one occasion or a series of injections over a period of hours, days, months or years with the regression or destruction of the tissue treated being monitored by means of biopsy, imaging or other methods of monitoring tissue growth. The injection into the tissue to be treated may be by a device inserted into an orifice such as the nose, mouth, ear, vagina, rectum or urethra or through an incision in order to reach the tissue in vivo and may performed in conjunction with an imaging or optical system such as ultrasound, fibre optic scope, x-rays, scans (including computerized axial tomography (CT), positron emission tomography (PET), and magnetic resonance imaging (MRI)), and contrast studies in order to identify the appropriate site for the injection(s). A device may be used to provide a constant infusion of Specific Peptide to the tissue over time.

A Specific Peptide may be administered alone, in combination or conjunction with another peptide, drug or compound or conjugated to a carrier or an antibody. The Specific Peptides may be administered intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transdermally, via an aerosol, infusion, bolus injection, implantation device, sustained release system, etc., alone, in combination or conjunction with another peptide, drug or compound or conjugated to a carrier or an antibody.

In another embodiment, a method is provided that comprises the administration of a composition containing one or more Specific Peptides as part of or in conjunction with a treatment or surgical procedure for a condition, such as the removal or reduction in size of a benign tumor. The condition for which the Specific Peptides may be administered may also be a hyperplasia, hypertrophy, or overgrowth of a tissue selected from the group consisting of lung, breast, stomach, pancreas, prostate, bladder, bone, ovary, skin, kidney, sinus, colon, intestine, stomach, rectum, esophagus, blood, brain and its coverings, spinal cord and its coverings, muscle, connective tissue, adrenal, parathyroid, thyroid, uterus, testis, pituitary, reproductive organs, liver, gall bladder, eye, ear, nose, throat, tonsils, mouth, and lymph nodes and lymphoid system. Other such conditions include virally, bacterially, or parasitically altered tissue selected from the group consisting of lung, breast, stomach, pancreas, prostate, bladder, bone, ovary, skin, kidney, sinus, colon, intestine, stomach, rectum, esophagus, blood, brain and its coverings, spinal cord and its coverings, muscle, connective tissue, adrenal, parathyroid, thyroid, uterus, testis, pituitary, reproductive organs, liver, gall bladder, eye, ear, nose, throat, tonsils, mouth, and lymph nodes and lymphoid system. The condition may also be a malformation or disorder of a tissue selected from the group consisting of lung, breast, stomach, pancreas, prostate, bladder, bone, ovary, skin, kidney, sinus, colon, intestine, stomach, rectum, esophagus, blood, brain and its coverings, spinal cord and its coverings, muscle, connective tissue, adrenal, parathyroid, thyroid, uterus, testis, pituitary, reproductive organs, liver, gall bladder, eye, ear, nose, throat, tonsils, mouth, and lymph nodes and lymphoid system. In particular, the condition may be tonsiliar hypertrophy, prostatic hyperplasia, psoriasis, eczema, dermatoses or hemorrhoids; a vascular disease, such as atherosclerosis or arteriosclerosis, or a vascular disorder, such as varicose veins, stenosis or restenosis of an artery or a stent; or a cosmetic modification to a tissue, such as skin, eye, ear, nose, throat, mouth, muscle, connective tissue, hair, or breast tissue, including breast reduction surgery or reductive mammaplasty.

Additional embodiments encompass methods for the administration of a Specific Peptide in conjunction with a surgical or similar procedure employed to physically excise, ablate or otherwise kill or destroy tumor or other tissue or cellular elements required or desired to be removed or destroyed wherein a Specific Peptide is administered to the immediate area(s) surrounding the area(s) where the tumor or other tissue was removed in order to destroy or impede the growth of any tumor cells or other cellular elements not removed or destroyed by the procedure

Methods employing therapeutic compositions of Specific Peptides are also contemplated as embodiments. Such compositions may comprise a therapeutically effective amount of a Specific Peptide in admixture with a pharmaceutically acceptable carrier. The carrier material may be water for injection, preferably supplemented with other materials common in solutions for administration to mammals. Typically, a Specific Peptide for therapeutic use will be administered in the form of a composition comprising purified peptide in conjunction with one or more physiologically acceptable carriers, excipients, or diluents. Neutral buffered saline or saline mixed with serum albumin are exemplary appropriate carriers. Preferably, the product is formulated as a lyophilizate using appropriate excipients (e.g., sucrose). Other standard carriers, diluents, and excipients may be included as desired. Compositions may also comprise buffers known to those having ordinary skill in the art with an appropriate range of pH values, including Tris buffer of about pH 7.0-8.5, or acetate buffer of about pH 4.0-5.5, which may further include sorbitol or a suitable substitute therefor.

Embodiments also include methods comprising the use of Specific Peptides conjugated or linked or bound to an antibody, antibody fragment, antibody-like molecule, or a molecule with a high affinity to a specific tissue marker, such as a cellular receptor, signal peptide or over-expressed enzyme, for targeting to the unwanted cellular elements. The antibody, antibody fragment, antibody-like molecule, or molecule with a high affinity to a specific tissue marker is used to target the Peptide conjugate to a specific cellular or tissue target, including undetected cancerous or pre-cancerous cells. For example, a tissue, gland or organ or a cancerous or pre-cancerous cell within such tissue, gland or organ with a distinctive surface antigen or expressed antigen may be targeted by the antibody, antibody fragment, or antibody-like binding molecule and the cells may be killed by the Peptide. Such an approach using antibody targeting has the anticipated advantages of decreasing dosage, increasing the likelihood of binding to and uptake by the target cells, and increased usefulness for targeting and treating small tissue sites or undetected cancers or pre-cancerous conditions.

Embodiments also include methods that use Specific Peptides conjugated or linked or bound to a protein or other molecule to form a composition that, upon cleavage at or near the site(s) of the unwanted cells by a site-specific enzyme or protease or by an antibody conjugate that targets unwanted cells, releases the Peptide at or near the site(s) of the unwanted cells

Embodiments also include methods that use Specific Peptides conjugated or linked or bound to a protein or other molecule to form a composition that releases the Peptide or some biologically active fragment of the Peptide upon exposure of the tissue to be treated to light (as in laser therapies or other photo-dynamic or photo-activated therapy), other forms of electromagnetic radiation such as infra-red radiation, ultraviolet radiation, x-ray or gamma ray radiation, localized heat, alpha or beta radiation, ultrasonic emissions, or other sources of localized energy.

The Specific Peptides may be employed alone, together, or in combination or conjunction with other pharmaceutical compositions, such as statins, COX-2 inhibitors, non-steriodal anti-inflammatory drugs (NSAIDs), cytokines, growth factors, antibiotics, apoptotis-inducing agents, anti-inflammatories, and/or chemotherapeutic agents as is appropriate for the type of cancer targeted for prevention or risk reduction. The Specific Peptides may be employed alone, together, or in combination or conjunction with other pharmaceutical compositions, compounds, vitamins, nutrients or trace elements, such as vitamin B6, vitamin C, vitamin D, vitamin E, folic acid, niacin, omega-3 fatty acids such as eicosapentaenoic acid (EPA) and docosahexaenoic acid (DHA), and selenium, as is appropriate for the type of cancer targeted for prevention or risk reduction.

Embodiments also include methods using therapeutic compositions of Specific Peptides employing dendrimers, fullerenes, and other synthetic molecules, polymers and macromolecules where the Peptide and/or its corresponding DNA molecule is conjugated with, attached to or enclosed in the molecule, polymer or macromolecule, either by itself or in conjunction with other species of molecule such as a tissue-specific or cancer cell marker. For example, U.S. Pat. No. 5,714,166, Bioactive and/or Targeted Dendimer Conjugates, provides a method of preparing and using, inter alia, dendritic polymer conjugates composed of at least one dendrimer with a target director(s) and at least one bioactive agent conjugated to it. The disclosure of U.S. Pat. No. 5,714,166 is incorporated by reference herein in its entirety.

Embodiments also include methods of using therapeutic compositions containing Specific Peptides and/or genes and drug delivery vehicles such as lipid emulsions, micelle polymers, polymer microspheres, electroactive polymers, hydrogels and liposomes.

Embodiments also include methods that transfer genes or gene equivalents encoding Specific Peptides to the unwanted cells. Over-expression of the Specific Peptide within the targeted tissue may be used to induce cells in the tissue to die and thus reduce the tissue cell population. The gene or gene equivalent transfer of the Specific Peptide to treat the unwanted cellular elements is anticipated to have the advantage of requiring less dosage, and of being passed on to the cellular progeny of the targeted cellular elements, thus necessitating less frequent therapy, and less total therapy. Embodiments also include the transfer of genes that code for a fusion protein containing a Specific Peptide to the unwanted cells or neighboring cells where, following the expression of the gene and the production and/or secretion of the fusion protein, the fusion protein is cleaved either by native enzymes or proteases or by a prodrug to release the Peptide in, at or near the unwanted cells.

Embodiments also include methods that use cloned recombinant peptide-antibody conjugates; cloned recombinant peptide-antibody fragment conjugates; and cloned recombinant peptide-antibody-like protein conjugates. The advantages of a cloned Specific Peptide combined with targeting conjugate (such as an antibody, antibody fragment, antibody-like molecule, or a molecule with a high affinity to a tissue-specific receptor or other tissue, pre-cancerous or cancer cell specific marker) are that such a molecule combines the targeting advantages described above in addition to advantages for manufacturing and standardized production of the cloned conjugated molecule.

Solid dosage forms for oral administration include, but are not limited to, capsules, tablets, pills, powders, and granules. In such solid dosage forms, the active compound is admixed with at least one of the following: (a) one or more inert excipients (or carrier), such as sodium citrate or dicalcium phosphate; (b) fillers or extenders, such as starches, lactose, sucrose, glucose, mannitol, and silicic acid; (c) binders, such as carboxymethylcellulose, alginates, gelatin, polyvinylpyrrolidone, sucrose and acacia; (d) humectants, such as glycerol; (a) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as acetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or mixtures thereof. For capsules, tablets, and pills, the dosage forms may also comprise buffering agents.

Liquid dosage forms for oral administration include pharmaceutically acceptable emulsions, solutions, suspensions, syrups, and elixirs. In addition to the active compounds, the liquid dosage forms may comprise inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, oils, such as cottonseed oil, groundnut oil, corn germ oil, olive oil, castor oil, and sesame oil, glycerol, tetrahydrofurfuryl alcohol, polyethyleneglycols, fatty acid esters of sorbitan, or mixtures of these substances, and the like.

Besides such inert diluents, the composition may also include adjuvants, such as wetting agents, emulsifying and suspending agents, sweetening, flavoring, and perfuming agents.

Actual dosage levels of active ingredients for use in the methods of the present embodiments may be varied to obtain an amount of Specific Peptide that is effective to obtain a desired therapeutic response for a particular composition and method of administration. The selected dosage level therefore depends upon the desired therapeutic effect, the route of administration, the desired duration of treatment, and other factors.

With mammals, including humans, the effective amounts for use in the methods described herein may be administered on the basis of body surface area. The interrelationship of dosages for animals of various sizes, species and humans (based on mg/M² of body surface) is described by E. J. Freireich et al., Cancer Chemother. Rep., 50 (4):219 (1966). Body surface area may be approximately determined from the height and weight of an individual (see e.g., Scientific Tables, Geigy Pharmaceuticals, Ardsley, N.Y. pp. 537-538 (1970)).

The total daily dose of the Specific Peptide administered to a host for use in the methods described herein may be in single or divided doses. Dosage unit compositions may contain such amounts of such submultiples thereof as may be used to make up the daily dose. It will be understood, however, that the specific dose level for any particular patient will depend upon a variety of factors including the body weight, general health, sex, diet, time and route of administration, potency of the administered drug, rates of absorption and excretion, combination with other drugs and the severity of the particular disease being treated.

Embodiments also include methods of administering a Specific Peptide composition that includes, but is not limited to, administering the compounds intramuscularly, orally, intravenously, intraperitoneally, intracerebrally (intraparenchymally), intracerebroventricularly, intratumorally, intralesionally, intradermally, intrathecally, intranasally, intraocularly, intraarterially, topically, transdermally, via an aerosol, infusion, bolus injection, implantation device, sustained release system etc.

Embodiments also include methods of administering a Specific Peptide by a transdermal or transcutaneous route. One example of such an embodiment is the use of a patch. In particular, a patch may be prepared with a fine suspension of Peptide in, for example, dimethylsulfoxide (DMSO), or a mixture of DMSO with cottonseed oil and brought into contact with the skin away from the tissue site inside a skin pouch. Other mediums or mixtures thereof with other solvents and solid supports would work equally as well. The patch may contain the Peptide compound in the form of a solution or a suspension. The patch may then be applied to the skin of the patient, for example, by means of inserting it into a skin pouch of the patient formed by folding and holding the skin together by means of stitches, clips or other holding devices. This pouch should be employed in such a manner so that continuous contact with the skin is assured without the interference of the mammal. Besides using a skin pouch, any device may be used which ensures the firm placement of the patch in contact with the skin. For instance, an adhesive bandage could be used to hold the patch in place on the skin.

Embodiments also include methods of administering a Specific Peptide in a sustained release formulation or preparation. Suitable examples of sustained-release preparations include semipermeable polymer matrices in the form of shaped articles, e.g. films, or microcapsules. Sustained release matrices include polyesters, polyethylene glycol and its derivatives, hydrogels, polylactides (U.S. Pat. No. 3,773,919, EP 58,481), copolymers of L-glutamic acid and gamma ethyl-L-glutamate (Sidman et al., Biopolymers, 22: 547-556), poly(2-hydroxyethyl-methacrylate) (Langer et al., J. Biomed. Mater. Res., 15: 167-277 and Langer, Chem. Tech., 12: 98-105), ethylene vinyl acetate (Langer et al., supra) or poly-D(−)-3-hydroxybutyric acid (EP 133,988). Sustained-release compositions also may include liposomes, which may be prepared by any of several methods known in the art (e.g., Eppstein et al., Proc. Natl. Acad. Sci. USA, 82: 3688-3692; EP 36,676; EP 88,046; and EP 143,949).

Other embodiments include methods of administering a Specific Peptide by implantation of a device within the tissue, gland, organ or cellular mass to be treated. One example of such an embodiment is the implantation of a wafer containing Peptide in the tissue to be treated. The wafer releases a therapeutic dose of Peptide into the tissue over time. Alternatively or additionally, the composition may be administered locally via implantation into the affected area of a membrane, sponge, or other appropriate material on to which the Specific Peptide has been absorbed. Where an implantation device is used, the device may be implanted into any suitable tissue or organ, and delivery of the Peptide may be directly through the device via bolus, or via continuous administration, or via catheter using continuous infusion.

Another method in accordance with additional embodiments, is to introduce one or more copies of a Specific Peptide-encoding gene into the cell being targeted and, if necessary, inducing the copy(ies) of the gene to begin producing Peptide intracellularly. One manner in which gene therapy may be applied is to use the Specific Peptide-encoding gene (either genomic DNA, cDNA, and/or synthetic DNA encoding the Peptide (or a fragment, variant, homologue or derivative thereof)) which may be operably linked to a constitutive or inducible promoter to form a gene therapy DNA construct. The promoter may be homologous or heterologous to an endogenous Peptide-encoding gene, provided that it is active in the cell or tissue type into which the construct will be inserted. Other components of the gene therapy DNA construct may optionally include, as required, DNA molecules designed for site-specific integration (e.g., endogenous flanking sequences useful for homologous recombination), tissue-specific promoter, enhancer(s) or silencer(s), DNA molecules capable of providing a selective advantage over the parent cell, DNA molecules useful as labels to identify transformed cells, negative selection systems, cell specific binding agents (as, for example, for cell targeting) cell-specific internalization factors, and transcription factors to enhance expression by a vector as well as factors to enable vector manufacture.

Means of gene delivery to a cell or tissue in vivo or ex vivo include (but are not limited to) direct injection of bare DNA, ballistic methods, liposome-mediated transfer, receptor-mediated transfer (ligand-DNA complex), electroporation, and calcium phosphate precipitation. See U.S. Pat. Nos. 4,970,154, WO 96/40958, U.S. Pat. No. 5,679,559, U.S. Pat. No. 5,676,954, and U.S. Pat. No. 5,593,875, the disclosures of each of which are incorporated by reference herein in their entirety. Means of gene delivery to a cell or tissue in vivo or ex vivo may also include use of a viral vector such as a retrovirus, adenovirus, adeno-associated virus, pox virus, lentivirus, papilloma virus or herpes simplex virus, use of a DNA-protein conjugate and use of a liposome. The use of gene therapy vectors is described, for example, in U.S. Pat. Nos. 5,672,344, U.S. Pat. No. 5,399,346, U.S. Pat. No. 5,631,236, and U.S. Pat. No. 5,635,399, the disclosures of each of which are incorporated by reference herein in their entirety.

The methods of embodiments also include the delivery of Specific Peptide-encoding gene(s) through implanting into patients certain cells that have been genetically engineered ex vivo, using methods such as those described herein, to express and secrete the Specific Peptide. Such cells may be animal or human cells, and may be derived from the patient's own tissue or from another source, either human or non-human. Optionally, the cells may be immortalized or be stem cells. In order to decrease the chance of an immunological response, however, it is preferred that the cells be encapsulated to avoid infiltration of surrounding tissues. The encapsulation materials are typically biocompatible, semi-permeable polymeric enclosures or membranes that allow release of the protein product(s) but prevent destruction of the cells by the patient's immune system or by other detrimental factors from the surrounding tissues. Methods used for membrane encapsulation of cells are familiar to the skilled artisan, and preparation of encapsulated cells and their implantation in patients may be accomplished without undue experimentation. See, e.g., U.S. Pat. Nos. 4,892,538; 5,011,472; and 5,106,627, the disclosures of each of which are incorporated by reference herein in their entirety. A system for encapsulating living cells is described in PCT WO 91/10425. Techniques for formulating a variety of other sustained or controlled delivery means, such as liposome carriers, bio-erodible particles or beads, are also known to those in the art, and are described, for example, in U.S. Pat. No. 5,653,975, the disclosure of which is incorporated by reference herein in their entirety. The cells, with or without encapsulation, may be implanted into suitable body tissues or organs of the patient.

Methods of making proteins and peptides such as the Specific Peptides are well known to those skilled in the arts. Some of these methods are disclosed in pending U.S. patent application Ser. No. 10/153,334, entitled: Peptides Effective In The Treatment Of Tumors And Other Conditions Requiring The Removal Or Destruction Of Cells; Ser. No. 10/198,069, entitled: Peptides Effective In The Treatment Of Tumors And Other Conditions Requiring The Removal Or Destruction Of Cells; Ser. No. 10/198,070, entitled: Peptides Effective In The Treatment Of Tumors And Other Conditions Requiring The Removal Or Destruction Of Cells, Ser. No. 10/294,891 entitled: Peptides Effective In The Treatment Of Tumors And Other Conditions Requiring The Removal Or Destruction Of Cells; Ser. No. 10/920,313 entitled: Peptides Effective In The Treatment Of Tumors And Other Conditions Requiring The Removal Or Destruction Of Cells; and Ser. No. 11/680,119 entitled: Peptides Effective In The Treatment Of Tumors And Other Conditions Requiring The Removal Or Destruction Of Cells, the disclosures of each of which are incorporated by reference herein in their entirety.

Throughout this description, including the foregoing description of related art, any and all publicly available documents described herein, including any and all U.S. patents or patent applications, are specifically incorporated by reference herein in their entirety. The foregoing description of related art is not intended in any way as an admission that any of the documents described therein, including pending U.S. patent applications, are prior art to the present embodiments. Moreover, the description herein of any disadvantages associated with the described products, methods, and/or apparatus, is not intended to limit the invention. Indeed, aspects of embodiments may include certain features of the described products, methods, and/or apparatus without suffering from their described disadvantages.

Both the foregoing general description and the following detailed description are exemplary and explanatory and are intended to provide further explanation of the invention as claimed. Other objects, advantages, and features will be readily apparent to those skilled in the art from the following detailed description of the embodiments.

This disclosure expressly incorporates by reference the examples contained in pending U.S. patent application Ser. No. 10/092,934, Methods of Treating Tumors and Related Conditions Using Neural Thread Proteins, which reveal that the whole AD7c-protein is an effective agent for causing cell death both in vitro in glioma and neuroblastoma cell cultures and in vivo in normal rodent muscle tissue, subcutaneous connective tissue, and dermis and in a variety of different human and non-human origin tumors, including mammary carcinoma, skin carcinoma and papilloma, colon carcinoma, glioma of brain, and others in rodent models. This application also expressly incorporates by reference the examples contained in pending U.S. patent application Ser. No. 10/153,334, entitled: Peptides Effective In The Treatment Of Tumors And Other Conditions Requiring The Removal Or Destruction Of Cells; Ser. No. 10/198,069, entitled: Peptides Effective In The Treatment Of Tumors And Other Conditions Requiring The Removal Or Destruction Of Cells; Ser. No. 10/198,070, entitled: Peptides Effective In The Treatment Of Tumors And Other Conditions Requiring The Removal Or Destruction Of Cells, Ser. No. 10/294,891 entitled: Peptides Effective In The Treatment Of Tumors And Other Conditions Requiring The Removal Or Destruction Of Cells; Ser. No. 10/920,313 entitled: Peptides Effective In The Treatment Of Tumors And Other Conditions Requiring The Removal Or Destruction Of Cells; and Ser. No. 11/680,119 entitled: Peptides Effective In The Treatment Of Tumors And Other Conditions Requiring The Removal Or Destruction Of Cells, each of which reveal that certain peptides specified therein are effective agents for causing cell death in vivo in normal rodent muscle tissue, subcutaneous connective tissue, dermis and other tissue.

It will be apparent to those skilled in the art that various modifications and variations can be made in the methods and compositions of the disclosed embodiments without departing from the spirit or scope thereof. Throughout the specification, any and all references to a publicly available document, including a U.S. patent, are specifically incorporated by reference.

What is claimed is:

1. A method of preventing or reducing the risk or incidence of cancer in a tissue, gland, organ, or other cellular mass of a mammal comprising administering to a mammal at least one compound comprising an isolated peptide consisting of a Specific Peptide selected from the group consisting of the amino acid sequence of any one of SEQ ID NO: 1 to 116.

2. The method of claim 1, wherein the mammal is human.

3. The method of claim 1, wherein the cancer is breast cancer, prostate cancer, oropharyngeal cancer, lymphoma, thyroid cancer, ovarian cancer, lung cancer, colon cancer, or stomach cancer.

4. The method of claim 1, wherein the mammal has an increased susceptibility to or risk of acquiring cancer.

5. The method of claim 1, wherein the mammal has increased risk factor(s) for prostate cancer.

6. The method of claim 1, wherein the mammal has an elevated Prostate-Specific Antigen (PSA) level.

7. The method of claim 1, wherein the mammal has an increased susceptibility or risk of acquiring cancer as a result of a genetic mutation, polymorphism or condition.

8. The method of claim 1, wherein the mammal is at increased risk of acquiring breast or ovarian cancer.

9. The method of claim 8, wherein the mammal further has the BRCA1 genetic mutation.

10. The method of claim 8, wherein the mammal further has the BRCA2 genetic mutation.

11. The method of claim 1, wherein the mammal has an increased susceptibility or risk of acquiring cancer as a result of exposure to, contact with or ingestion of a carcinogenic agent.

12. The method of claim 11, wherein the carcinogenic agent is a form of radiation, known carcinogenic chemical agent, infectious agent, or pharmaceutical agent.

13. The method of claim 1, wherein the compound is administered in conjunction with another compound or drug.

14. The method of claim 1, wherein the cancer is prostate cancer, and wherein the at least one compound is administered in conjunction with selenium, vitamin C or E, or eicosapentaenoic acid (EPA) or docosahexaenoic acid (DHA).

15. The method of claim 1, wherein the isolated peptide comprises an amino acid in a reverse-D order based on the amino acid sequence for a peptide consisting of the amino acid sequence of any one of SEQ ID NO: 1 to 116.

16. The method of claim 1, wherein the isolated peptide consists of the amino acid sequence of any one of SEQ ID NO: 1 to 116 and at least one and up to 25 additional amino acids flanking either the 3′ or 5′ end of the peptide.

17. The method of claim 1, wherein the isolated peptide comprises at least two peptides consisting of the amino acid sequence of any one of SEQ ID NO: 1 to 116.

18. The method of claim 1, wherein the isolated peptide comprises at least two repetitions of a peptide consisting of the amino acid sequence of any one of SEQ ID NO: 1 to 116.

19. The method of claim 1, wherein the isolated peptide is a mimetic of a peptide consisting of the amino acid sequence of any one of SEQ ID NO: 1 to 116.

20. The method of claim 1, wherein the isolated peptide comprises a peptide consisting of the amino acid sequence of any one of SEQ ID NO: 1 to 116 fused to an antibody, fragment of an antibody, or an antibody-like molecule.

21. A method of preventing or reducing the risk of cancer in a mammal comprising administering to the mammal a therapeutically effective amount of an isolated peptide consisting of a Specific Peptide selected from the group consisting of the amino acid sequence of any one of SEQ ID NO: 1 to 116.

22. The method of claim 21, wherein the peptide is administered by a method selected from the group consisting of orally, subcutaneously, intradermally, intranasally, intravenously, intramuscularly, intrathecally, intranasally, intratumorally, topically, transdermally, intraperitoneally, intracerebrally (intraparenchymally), intracerebroventricularly, intratumorally, intralesionally, intraocularly, and intraarterially.

23. The method of claim 21, wherein the peptide is administered into or in close proximity to the tissue, gland, organ or cellular mass at risk for cancer.

24. The method of claim 21, wherein the tissue, gland or organ treated is selected from the group consisting of lung, breast, stomach, pancreas, prostate, bladder, bone, ovary, skin, kidney, sinus, colon, intestine, stomach, rectum, esophagus, heart, spleen, salivary gland, blood, brain and its coverings, spinal cord and its coverings, muscle, connective tissue, adrenal, parathyroid, thyroid, uterus, testis, pituitary, reproductive organs, liver, gall bladder, eye, ear, nose, throat, tonsils, mouth, and lymph nodes and lymphoid system.


SEQUENCE LISTING (117 sequences)

SEQ ID NO: 1 - protein, 15 aa, Artificial Sequence
  Description of Artificial Sequence Synthetic peptide
MEFSLLLPRLECNGA

SEQ ID NO: 2 - protein, 15 aa, Artificial Sequence
  Description of Artificial Sequence Synthetic peptide
GAISAHRNLRLPGSS

SEQ ID NO: 3 - protein, 17 aa, Artificial Sequence
  Description of Artificial Sequence Synthetic peptide
DSPASASPVAGITGMCT

SEQ ID NO: 4 - protein, 16 aa, Artificial Sequence
  Description of Artificial Sequence Synthetic peptide
MCTHARLILYFFLVEM

SEQ ID NO: 5 - protein, 11 aa, Artificial Sequence
  Description of Artificial Sequence Synthetic peptide
YFFLVEMEFLH

SEQ ID NO: 6 - protein, 11 aa, Artificial Sequence
  Description of Artificial Sequence Synthetic peptide
VGQAGLELPTS

SEQ ID NO: 7 - protein, 17 aa, Artificial Sequence
  Description of Artificial Sequence Synthetic peptide
DDPSVSASQSARYRTGH

SEQ ID NO: 8 - protein, 14 aa, Artificial Sequence
  Description of Artificial Sequence Synthetic peptide
TGHHARLCLANFCG

SEQ ID NO: 9 - protein, 17 aa, Artificial Sequence
  Description of Artificial Sequence Synthetic peptide
ANFCGRNRVSLMCPSWS

SEQ ID NO: 10 - protein, 19 aa, Artificial Sequence
  Description of Artificial Sequence Synthetic peptide
PELKQSTCLSLPKCWDYRR

SEQ ID NO: 11 - protein, 17 aa, Artificial Sequence
  Description of Artificial Sequence Synthetic peptide
LKQSTCLSLPKCWDYRR

SEQ ID NO: 12 - protein, 14 aa, Artificial Sequence
  Description of Artificial Sequence Synthetic peptide
STCLSLPKCWDYRR

SEQ ID NO: 13 - protein, 11 aa, Artificial Sequence
  Description of Artificial Sequence Synthetic peptide
LSLPKCWDYRR

SEQ ID NO: 14 - protein, 13 aa, Artificial Sequence
  Description of Artificial Sequence Synthetic peptide
KCWDYRRAAVPGL

SEQ ID NO: 15 - protein, 19 aa, Artificial Sequence
  Description of Artificial Sequence Synthetic peptide
KCWDYRRAAVPGLFILFFL

SEQ ID NO: 16 - protein, 24 aa, Artificial Sequence
  Description of Artificial Sequence Synthetic peptide
KCWDYRRAAVPGLFILFFLRHRCP

SEQ ID NO: 17 - protein, 38 aa, Artificial Sequence
  Description of Artificial Sequence Synthetic peptide
KCWDYRRAAVPGLFILFFLRHRCPTLTQDEVQWCDHSS

SEQ ID NO: 18 - protein, 5 aa, Artificial Sequence
  Description of Artificial Sequence Synthetic peptide
WDYRR

SEQ ID NO: 19 - protein, 13 aa, Artificial Sequence
  Description of Artificial Sequence Synthetic peptide
FILFFLRHRCPTL

SEQ ID NO: 20 - protein, 25 aa, Artificial Sequence
  Description of Artificial Sequence Synthetic peptide
FILFFLRHRCPTLTQDEVQWCDHSS

SEQ ID NO: 21 - protein, 29 aa, Artificial Sequence
  Description of Artificial Sequence Synthetic peptide
HRCPTLTQDEVQWCDHSSLQPSTPEIKHP

SEQ ID NO: 22 - protein, 14 aa, Artificial Sequence
  Description of Artificial Sequence Synthetic peptide
PASASQVAGTKDMH

SEQ ID NO: 23 - protein, 18 aa, Artificial Sequence
  Description of Artificial Sequence Synthetic peptide
DMHHYTWLIFIFIFNFLR

SEQ ID NO: 24 - protein, 19 aa, Artificial Sequence [sequence omitted]

SEQ ID NO: 25 - protein, 45 aa, Artificial Sequence
  Description of Artificial Sequence Synthetic peptide
SVTQAGVQWRNLGSLQPLPPGFKLFSCPSLLSSWDYRRPPRLANF

SEQ ID NO: 26 - protein, 19 aa, Artificial Sequence
  Description of Artificial Sequence Synthetic peptide
PGFKLFSCPSLLSSWDYRR

SEQ ID NO: 27 - protein, 24 aa, Artificial Sequence
  Description of Artificial Sequence Synthetic peptide
FKLFSCPSLLSSWDYRRPPRLANF

SEQ ID NO: 28 - protein, 14 aa, Artificial Sequence
  Description of Artificial Sequence Synthetic peptide
FSCPSLLSSWDYRR

SEQ ID NO: 29 - protein, 10 aa, Artificial Sequence
  Description of Artificial Sequence Synthetic peptide
SLLSSWDYRR

SEQ ID NO: 30 - protein, 5 aa, Artificial Sequence
  Description of Artificial Sequence Synthetic peptide
SSWDY

SEQ ID NO: 31 - protein, 7 aa, Artificial Sequence
  Description of Artificial Sequence Synthetic peptide
SSWDYRR

SEQ ID NO: 32 - protein, 25 aa, Artificial Sequence
  Description of Artificial Sequence Synthetic peptide
SSWDYRRPPRLANFFVFLVEMGFTM

SEQ ID NO: 33 - protein, 11 aa, Artificial Sequence
  Description of Artificial Sequence Synthetic peptide
FVFLVEMGFTM

SEQ ID NO: 34 - protein, 23 aa, Artificial Sequence
  Description of Artificial Sequence Synthetic peptide
MGFTMFARLILISGPCDLPASAS

SEQ ID NO: 35 - protein, 5 aa, Artificial Sequence
  Description of Artificial Sequence Synthetic peptide
ISGPC

SEQ ID NO: 36 - protein, 17 aa, Artificial Sequence
  Description of Artificial Sequence Synthetic peptide
DLPASASQSAGITGVSH

SEQ ID NO: 37 - protein, 17 aa, Artificial Sequence
  Description of Artificial Sequence Synthetic peptide
GVSHHARLIFNFCLFEM

SEQ ID NO: 38 - protein, 10 aa, Artificial Sequence
  Description of Artificial Sequence Synthetic peptide
NFCLFEMESH

SEQ ID NO: 39 - protein, 46 aa, Artificial Sequence
  Description of Artificial Sequence Synthetic peptide
SVTQAGVQWPNLGSLQPLPPGLKRFSCLSLPSSWDYGHLPPHPANF

SEQ ID NO: 40 - protein, 19 aa, Artificial Sequence
  Description of Artificial Sequence Synthetic peptide
PPGLKRFSCLSLPSSWDYG

SEQ ID NO: 41 - protein, 14 aa, Artificial Sequence
  Description of Artificial Sequence Synthetic peptide
FSCLSLPSSWDYGH

SEQ ID NO: 42 - protein, 9 aa, Artificial Sequence
  Description of Artificial Sequence Synthetic peptide
LSLPSSWDY

SEQ ID NO: 43 - protein, 37 aa, Artificial Sequence
  Description of Artificial Sequence Synthetic peptide
SSWDYGHLPPHPANFCIFIRGGVSPYLSGWSQTPDLR

SEQ ID NO: 44 - protein, 20 aa, Artificial Sequence
  Description of Artificial Sequence Synthetic peptide
PGFFKLFSCPSLLSSWDYRR

SEQ ID NO: 45 - protein, 19 aa, Artificial Sequence
  Description of Artificial Sequence Synthetic peptide
PELKQSTCLSLPKCWDYRR

SEQ ID NO: 46 - protein, 19 aa, Artificial Sequence
  Description of Artificial Sequence Synthetic peptide
PPGLKRFSCLSLPSSWDYG

SEQ ID NO: 47 - protein, 14 aa, Artificial Sequence
  Description of Artificial Sequence Synthetic peptide
FSCLSLPSSWDYGH

SEQ ID NO: 48 - protein, 14 aa, Artificial Sequence
  Description of Artificial Sequence Synthetic peptide
STCLSLPKCWDYRR

SEQ ID NO: 49 - protein, 14 aa, Artificial Sequence
  Description of Artificial Sequence Synthetic peptide
FSCPSLLSSWDYRR

SEQ ID NO: 50 - protein, 9 aa, Artificial Sequence
  Description of Artificial Sequence Synthetic peptide
LSLPSSWDY

SEQ ID NO: 51 - protein, 11 aa, Artificial Sequence
  Description of Artificial Sequence Synthetic peptide
LSLPKCWDYRR

SEQ ID NO: 52 - protein, 10 aa, Artificial Sequence
  Description of Artificial Sequence Synthetic peptide
SLLSSWDYRR

SEQ ID NO: 53 - protein, 9 aa, Artificial Sequence
  Description of Artificial Sequence Synthetic peptide
LPSSWDYRR

SEQ ID NO: 54 - protein, 7 aa, Artificial Sequence
  Description of Artificial Sequence Synthetic peptide
SSWDYRR

SEQ ID NO: 55 - protein, 5 aa, Artificial Sequence
  Description of Artificial Sequence Synthetic peptide
SSWDY

SEQ ID NO: 56 - protein, 13 aa, Artificial Sequence
  Description of Artificial Sequence Synthetic peptide
SSWDYRRFILFFL

SEQ ID NO: 57 - protein, 11 aa, Artificial Sequence
  Description of Artificial Sequence Synthetic peptide
WDYRRFIFNFL

SEQ ID NO: 58 - protein, 6 aa, Artificial Sequence
  Description of Artificial Sequence Synthetic peptide
FNFCLF

SEQ ID NO: 59 - protein, 6 aa, Artificial Sequence
  Description of Artificial Sequence Synthetic peptide
FIFNFL

SEQ ID NO: 60 - protein, 13 aa, Artificial Sequence
  Description of Artificial Sequence Synthetic peptide
PASASPVAGITGM

SEQ ID NO: 61 - protein, 13 aa, Artificial Sequence
  Description of Artificial Sequence Synthetic peptide
PASASQVAGTKDM

SEQ ID NO: 62 - protein, 13 aa, Artificial Sequence
  Description of Artificial Sequence Synthetic peptide
PASASQSAGITGV

SEQ ID NO: 63 - protein, 9 aa, Artificial Sequence
  Description of Artificial Sequence Synthetic peptide
PASASPVAG

SEQ ID NO: 64 - protein, 6 aa, Artificial Sequence
  Description of Artificial Sequence Synthetic peptide
FFLVEM

SEQ ID NO: 65 - protein, 9 aa, Artificial Sequence
  Description of Artificial Sequence Synthetic peptide
SVTQAGVQW

SEQ ID NO: 66 - protein, 17 aa, Artificial Sequence
  Description of Artificial Sequence Synthetic peptide
IDQQVLSRIKLEIKRCL

SEQ ID NO: 67 - protein, 9 aa, Artificial Sequence
  Description of Artificial Sequence Synthetic peptide
LSRIKLEIK

SEQ ID NO: 68 - protein, 22 aa, Artificial Sequence
  Description of Artificial Sequence Synthetic peptide
GDHGRPNLSRLKLAIKYEVKKM

SEQ ID NO: 69 - protein, 16 aa, Artificial Sequence
  Description of Artificial Sequence Synthetic peptide
QQSIAVKFLAVFGVSI

SEQ ID NO: 70 - protein, 21 aa, Artificial Sequence
  Description of Artificial Sequence Synthetic peptide
GLLFPVFSVCYLIAPKSPLGL

SEQ ID NO: 71 - protein, 15 aa, Artificial Sequence
  Description of Artificial Sequence Synthetic peptide
MMVCWNRFGKWVYFI

SEQ ID NO: 72 - protein, 15 aa, Artificial Sequence
  Description of Artificial Sequence Synthetic peptide
SAIFNFGPRYLYHGV

SEQ ID NO: 73 - protein, 15 aa, Artificial Sequence
  Description of Artificial Sequence Synthetic peptide
PFYFLILVRIISFLI

SEQ ID NO: 74 - protein, 15 aa, Artificial Sequence
  Description of Artificial Sequence Synthetic peptide
GDMEDVLLNCTLLKR

SEQ ID NO: 75 - protein, 15 aa, Artificial Sequence
  Description of Artificial Sequence Synthetic peptide
SSRFRFWGALVCSMD

SEQ ID NO: 76 - protein, 15 aa, Artificial Sequence
  Description of Artificial Sequence Synthetic peptide
SCRFSRVAVTYRFIT

SEQ ID NO: 77 - protein, 15 aa, Artificial Sequence
  Description of Artificial Sequence Synthetic peptide
LLNIPSPAVWMARNT

SEQ ID NO: 78 - protein, 15 aa, Artificial Sequence
  Description of Artificial Sequence Synthetic peptide
MAQSRLTATSASRVQ

SEQ ID NO: 79 - protein, 15 aa, Artificial Sequence
  Description of Artificial Sequence Synthetic peptide
AILLSQPPKQLGLRA

SEQ ID NO: 80 - protein, 15 aa, Artificial Sequence
  Description of Artificial Sequence Synthetic peptide
PANTPLIFVFSLEAG

SEQ ID NO: 81 - protein, 15 aa, Artificial Sequence
  Description of Artificial Sequence Synthetic peptide
FHHICQAGLKLLTSG

SEQ ID NO: 82 - protein, 15 aa, Artificial Sequence
  Description of Artificial Sequence Synthetic peptide
DPPASAFQSAGITGV

SEQ ID NO: 83 - protein, 15 aa, Artificial Sequence
  Description of Artificial Sequence Synthetic peptide
SHLTQPANLDKKICS

SEQ ID NO: 84 - protein, 22 aa, Artificial Sequence
  Description of Artificial Sequence Synthetic peptide
NGGSCYVAQAGLKLLASCNPSK

SEQ ID NO: 85 - protein, 15 aa, Artificial Sequence
  Description of Artificial Sequence Synthetic peptide
MWTLKSSLVLLLCLT

SEQ ID NO: 86 - protein, 15 aa, Artificial Sequence
  Description of Artificial Sequence Synthetic peptide
CSYAFMFSSLRQKTS

SEQ ID NO: 87 - protein, 15 aa, Artificial Sequence
  Description of Artificial Sequence Synthetic peptide
EPQGKVPCGEHFRIR

SEQ ID NO: 88 - protein, 15 aa, Artificial Sequence
  Description of Artificial Sequence Synthetic peptide
QNLPEHTQGWLGSKW

SEQ ID NO: 89 - protein, 15 aa, Artificial Sequence
  Description of Artificial Sequence Synthetic peptide
LWLLFAVVPFVILKC

SEQ ID NO: 90 - protein, 15 aa, Artificial Sequence
  Description of Artificial Sequence Synthetic peptide
QRDSEKNKVRMAPFF

SEQ ID NO: 91 - protein, 16 aa, Artificial Sequence
  Description of Artificial Sequence Synthetic peptide
LHHIDSISGVSGKRMF

SEQ ID NO: 92 - protein, 15 aa, Artificial Sequence
  Description of Artificial Sequence Synthetic peptide
EAYYTMLHLPTTNRP

SEQ ID NO: 93 - protein, 15 aa, Artificial Sequence
  Description of Artificial Sequence Synthetic peptide
KIAHCILFNQPHSPR

SEQ ID NO: 94 - protein, 15 aa, Artificial Sequence
  Description of Artificial Sequence Synthetic peptide
SNSHSHPNPLKLHRR

SEQ ID NO: 95 - protein, 15 aa, Artificial Sequence
  Description of Artificial Sequence Synthetic peptide
SHSHNRPRAYILITI

SEQ ID NO: 96 - protein, 15 aa, Artificial Sequence
  Description of Artificial Sequence Synthetic peptide
LPSKLKLRTHSQSHH

SEQ ID NO: 97 - protein, 23 aa, Artificial Sequence
  Description of Artificial Sequence Synthetic peptide
NPLSRTSNSTPTNSFLMTSSKPR

SEQ ID NO: 98 - protein, 15 aa, Artificial Sequence
  Description of Artificial Sequence Synthetic peptide
SSSLGLPKCWDYRHE

SEQ ID NO: 99 - protein, 15 aa, Artificial Sequence
  Description of Artificial Sequence Synthetic peptide
LLSLALMINFRVMAC

SEQ ID NO: 100 - protein, 15 aa, Artificial Sequence
  Description of Artificial Sequence Synthetic peptide
TFKQHIELRQKISIV

SEQ ID NO: 101 - protein, 15 aa, Artificial Sequence
  Description of Artificial Sequence Synthetic peptide
PRKLCCMGPVCPVKI

SEQ ID NO: 102 - protein, 15 aa, Artificial Sequence
  Description of Artificial Sequence Synthetic peptide
ALLTINGHCTWLPAS

SEQ ID NO: 103 - protein, 15 aa, Artificial Sequence
  Description of Artificial Sequence Synthetic peptide
MFVFCLILNREKIKG

SEQ ID NO: 104 - protein, 15 aa, Artificial Sequence
  Description of Artificial Sequence Synthetic peptide
GNSSFFLLSFFFSFQ

SEQ ID NO: 105 - protein, 15 aa, Artificial Sequence
  Description of Artificial Sequence Synthetic peptide
NCCQCFQCRTTEGYA

SEQ ID NO: 106 - protein, 23 aa, Artificial Sequence
  Description of Artificial Sequence Synthetic peptide
VECFYCLVDKAAFECWWFYSFDT

SEQ ID NO: 107 - protein, 15 aa, Artificial Sequence
  Description of Artificial Sequence Synthetic peptide
MEPHTVAQAGVPQHD

SEQ ID NO: 108 - protein, 15 aa, Artificial Sequence
  Description of Artificial Sequence Synthetic peptide
LGSLQSLLPRFKRFS

SEQ ID NO: 109 - protein, 15 aa, Artificial Sequence
  Description of Artificial Sequence Synthetic peptide
CLILPKIWDYRNMNT

SEQ ID NO: 110 - protein, 16 aa, Artificial Sequence
  Description of Artificial Sequence Synthetic peptide
ALIKRNRYTPETGRKS

SEQ ID NO: 111 - protein, 9 aa, Artificial Sequence
  Description of Artificial Sequence Synthetic peptide
IDQQVLSRI

SEQ ID NO: 112 - protein, 8 aa, Artificial Sequence
  Description of Artificial Sequence Synthetic peptide
KLEIKRCL

SEQ ID NO: 113 - protein, 6 aa, Artificial Sequence
  Description of Artificial Sequence Synthetic peptide
VLSRIK

SEQ ID NO: 114 - protein, 7 aa, Artificial Sequence
  Description of Artificial Sequence Synthetic peptide
RIKLEIK

SEQ ID NO: 115 - protein, 13 aa, Artificial Sequence
  Description of Artificial Sequence Synthetic peptide
VLSRIKLEIKRCL

SEQ ID NO: 116 - protein, 13 aa, Artificial Sequence
  Description of Artificial Sequence Synthetic peptide
IDQQVLSRIKLEI

SEQ ID NO: 117 - protein, 5 aa, Artificial Sequence
  Description of Artificial Sequence Synthetic peptide
ETESH